(19) United States
(12) Patent Application Publication
(10) Pub. No.: US 2009/0005437 A1
(43) Pub. Date: Jan. 1, 2009

(54) Alpha-Ketoglutarates and Their Use as Therapeutic Agents

(76) Inventors: Eyal Gottlieb, Glasgow Strathclyde (GB); Mary A. Selak, Glasgow Strathclyde (GB); Elaine D. MacKenzie, Glasgow Strathclyde (GB); David G. Watson, Glasgow Strathclyde (GB)

Correspondence Address: NIXON & VANDERHYE, PC, 901 NORTH GLEBE ROAD, 11TH FLOOR, ARLINGTON, VA, 22203, US

(21) Appl. No.: 11/658,452

(22) PCT Filed: Aug. 9, 2005
(86) PCT No.: PCT/GB2005/003119

§ 371 Date: Jan. 26, 2007

(30) Foreign Application Priority Data
Aug. 9, 2004 (GB) 0417715.0
Oct. 1, 2004 (GB) 0421921.8

(51) Int. Cl. A61K 31/355; C07C 69/76; C07C 69/34; A61P 35/00; A61P 9/10; A61P 3/04; A61P 19/02; A61P 17/06; C07D 311/76; A61K 31/225

(52) U.S. Cl. 514/458; 560/76; 560/190; 549/407; 514/547; 435/375

(57) ABSTRACT

The present invention relates generally to the field of pharmaceuticals and medicine. More particularly, the present invention relates to certain compounds (e.g., α-ketoglutarate compounds; compounds that activate HIFα hydroxylase; compounds that increases the level of α-ketoglutarate, etc.) and their use in medicine, for example, in the treatment of cancer (e.g., cancer in which the activity of one of the enzymes in the tricarboxylic acid (TCA) cycle is down regulated), in the treatment of angiogenesis (e.g., hypoxia-induced angiogenesis). One preferred class of compounds are α-ketoglutarate compounds having a hydrophobic moiety that is, or is part of, an ester group formed from one of the acid groups of α-ketogluartic acid; and pharmaceutically acceptable salts, solvates, amides, esters, ethers, N-oxides, chemically protected forms, and prodrugs thereof.

### RELATED APPLICATIONS

This application is related to United Kingdom patent application GB 0417715.0 filed 9 Aug. 2004 and United Kingdom patent application GB 0421921.8 filed 1 Oct. 2004, the contents of each of which are incorporated herein by reference in their entirety.

### TECHNICAL FIELD

The present invention relates generally to the field of pharmaceuticals and medicine. More particularly, the present invention relates to certain compounds (e.g., α-ketoglutarate compounds; compounds that activate HIFα hydroxylase; compounds that increases the level of α-ketoglutarate, etc.) and their use in medicine, for example, in the treatment of cancer (e.g., cancer in which the activity of one of the enzymes in the tricarboxylic acid (TCA) cycle is down regulated), in the treatment of angiogenesis (e.g., hypoxia-induced angiogenesis), etc.

### BACKGROUND

A number of patents and publications are cited herein in order to more fully describe and disclose the invention and the state of the art to which the invention pertains. Full citations for these references are provided herein. Each of these references is incorporated herein by reference in its entirety into the present disclosure.

Throughout this specification, including any claims which follow, unless the context requires otherwise, the word “comprise,” and variations such as “comprises” and “comprising,” will be understood to imply the inclusion of a stated integer or step or group of integers or steps, but not the exclusion of any other integer or step or group of integers or steps.

It must be noted that, as used in the specification and any appended claims, the singular forms “a,” “an,” and “the” include plural referents unless the context clearly dictates otherwise. Thus, for example, reference to “a pharmaceutical excipient” includes mixtures of two or more such excipients, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Cancer is a serious disease and a major killer. Although there have been advances in the treatment of certain cancers in recent years, there is still a need for improvements in the treatment of the disease.

Cancer is characterised by the uncontrolled growth of cells due to cellular changes, which are mostly caused by inherited or somatic mutations of genes. The identification of such genes and the elucidation of the mechanism by which these genes affect the development of cancer is important in devising strategies of combating cancer.

Enzymes of the mitochondrial tricarboxylic acid (TCA) cycle have long been associated with cancer. Several mitochondrial proteins are tumour suppressors including succinate dehydrogenase (SDH) and fumarate hydratase (FH). Inherited or somatic mutations in subunits B, C or D of the SDH genes are associated with the development of phaeochromocytoma and paraganglioma (Baysal et al., 2000; Eng et al., 2003). Recently, other types of cancer have also been shown to carry or develop mutations in mitochondrial genes. For example, it has been shown that significant SDH down-regulation occurs in gastric and colorectal carcinoma, particularly during transition to the more aggressive Dukes' stage C, colorectal cancer, as compared to the confined Dukes' stage B tumours (Frederiksen et al., 2003; Habano et al., 2003).

Eng et al. (2003) discuss the link between mutations of gene encoding FH and SDH and cancer. The authors hypothesise that impaired mitochondrial function due to dysfunction of enzymes of the TCA cycle leads to severe energy deficiency and large amounts of oxygen free radicals. These radicals lead in turn to the induction of Hypoxia-inducible Factor-1α (HIF-1α) promoting cell proliferation or preventing apoptosis and thereby leading to neoplasia. The authors also suggest that mutant forms of SDH, which do not insert in the mitochondrial membrane, might have anti-apoptotic activity. However, the authors are unable to explain the mechanism underlying the anti-apoptotic activity.

Baysal (2003) suggests that SDH and FH could be involved in the control of cell proliferation under normal physiological conditions in the affected tissue types. However, the author provides no further suggestion regarding the mechanism of control.

Furthermore, tumours similar to phaeochromocytoma and paraganglioma are observed in the apparently unrelated von Hippel-Lindau (VHL) syndrome with a common feature of these tumours being elevated levels of HIF-1α (Eng et al., 2003, Pollard et al., 2003). Importantly, SDH or VHL mutations in these tumours are mutually exclusive (Eng et al., 2003).

Hypoxia-inducible factor-1 (HIF-1) is a heterodimer composed of an alpha (α) subunit and a beta (>subunit. (However, the terms “HIF-1” and “HIF-1α” are often used interchangeably to mean the complete protein, HIF-1). The beta subunit has been identified as the aryl hydrocarbon receptor nuclear translocator (ARNT/HIF-1β) and its protein level is unaffected by oxygen. Similar to HIF-1, HIF-1α is constitutively expressed regardless of the oxygenation state. However, under normoxic conditions this subunit is rapidly targeted for proteasome-mediated degradation via a protein-ubiquitin ligase complex containing the product of the von Hippel Lindau tumour suppressor protein (pVHL). pVHL recognizes the oxygen degradation domain (ODDD) of HIF-1α only under normoxic conditions. Following exposure to a hypoxic environment, this degradation pathway is blocked, allowing HIF-1α accumulation and subsequent movement to the nucleus where it activates hypoxia-responsive genes. In other words, the physiological function of HIF is to promote adaptation of cells to low oxygen by inducing neovascularization and glycolysis (Semenza et al., 2002; Pugh et al., 2003).

HIF-1α stability is controlled by HIFα prolyl hydroxylase (PHD) which hydroxylases two specific prolyl residues. More specifically, PHD hydroxylases the prolyl residues in the ODDD which regulate the binding of the pVHL to HIFα (Ivan et al., 2001; Jaakkola et al., 2001; Yu et al., 2001). Hydroxylation at the 4-position of Pro-402 and Pro-564 of HIFα (numbers refers to human HIF-1α) enables formation of two hydrogen bonds to pVHL and increases the binding of pVHL to HIFα by several orders of magnitude (Bruick et al., 2001; Epstein et al., 2001). This post-translational modification is catalyzed by the HIFα-prolyl hydroxylases (HPH1-3 or PHD1-3) (Bruick et al., 2001; Epstein et al., 2001; Ivan et al., 2002). PHD activity is dependent on molecular oxygen and is considered to be an important oxygen sensing mechanism in animal cells (Safran et al., 2003). In addition to oxygen, the PHDs utilize α-ketoglutarate as a co-substrate and require ferrous iron (Fe²⁺) and ascorbate as cofactors (Kaelin et al., 2002; Schofield et al., 1999). The PHD isozymes belong to the Fe²⁺- and α-ketoglutarate-dependent family of oxygenases that split molecular oxygen in order to hydroxylate their substrates and, in parallel, oxidize and decarboxylate α-ketoglutarate to succinate (Schofield et al., 1999).

WO 03/028663 discloses methods and compositions for assaying hypoxia-inducible factor prolyl hydroxylation to identify compounds that modulate the hydroxylation; however, the document fails to disclose any such compounds.

Although the events around the carcinogenic pathway involving HIF-1α stabilisation have been investigated, there are still numerous questions that remain unanswered.

In particular, a simple and effective way to inhibit HIF-1α stabilisation—and thereby inhibit the carcinogenic pathway—is still very much needed.

Furthermore, until now, there has been no clear indication or suggestion about how mutations in genes coding for enzymes of the TCA cycle might result in elevated levels of HIF-1α. Therefore, the range of treatment available for these cancers is limited. The primary treatment of pheochromocytomas and paragangliomas is surgical resection after appropriate medical hormonal blockade; Unresectable tumours may be treated with palliative chemotherapy with compounds such as cyclophosphamide, decarbazine, and vincristine, or external beam radiotherapy for bony metastases or ¹³¹I-labeled MIGB. However these therapies are either highly invasive or have large undesired side effects. Therefore, there remains a great need for treatments which are less invasive and which have little or no side effects. Preferably such a treatment would be tailor-made for the biochemical mechanism underlying these specific types of cancers.

Moreover, compounds that inhibit hypoxia-induced angiogenesis are still required as treatment for diseases that are characterised by this type of angiogenesis, including cancer.

The inventors have demonstrated how mutations and dysfunctions of genes and enzymes of the TCA cycle are linked to cancer. The inventors have developed strategies for treating cancer and have identified classes of compounds that are useful in these treatments.

For example, the inventors have demonstrated that inhibition of certain enzymes of the TCA cycle, such as SDH and FH, leads to the accumulation of succinate in cells. In turn, succinate inhibits the enzymatic activity of HIF-α prolyl hydroxylase (PHD) in the cytosol. Also, the inventors have demonstrated that α-ketoglutarate and α-ketoglutarate derivatives (e.g., esters) significantly enhance PHD activity under low oxygen conditions, thereby reducing HIF dramatically.

In other words, the inventors identified a new way of treating hypoxia-induced angiogenesis, which has useful pharmaceutical applications, for example, in the treatment of diseases that are characterised by hypoxia-induced angiogenesis.

### SUMMARY OF THE INVENTION

One aspect of the invention pertains to certain compounds (e.g., α-ketoglutarate compounds; compounds that activate HIFα hydroxylase; compounds that activate PHD; compounds that inhibit or prevent HIF stabilization; compounds that increases the level of α-ketoglutarate, etc.).

Another aspect of the invention pertains to a composition comprising an active compound as described herein and a pharmaceutically acceptable carrier or diluent.

Another aspect of the present invention pertains to a method of activating PHD in a cell, in vitro or in vivo, comprising contacting the cell with an effective amount of an active compound, as described herein.

Another aspect of the present invention pertains to a method of inhibiting or preventing HIF stabilization in a cell, in vitro or in vivo, comprising contacting the cell with an effective amount of an active compound, as described herein.

Another aspect of the present invention pertains to a method of activating HIFα hydroxylase (e.g., HIFα prolyl hydroxylase) in a cell, in vitro or in vivo, comprising contacting the cell with an effective amount of an active compound, as described herein. Another aspect of the present invention pertains to a method of (a) regulating (e.g., inhibiting) cell proliferation (e.g., proliferation of a cell), (b) inhibiting cell cycle progression, (c) promoting apoptosis, or (d) a combination of one or more these, in vitro or in vivo, comprising contacting cells (or the cell) with an effective amount of an active compound, as described herein.

Another aspect of the present invention pertains to an active compound, as described herein, for use in a method of treatment of the human or animal body by therapy.

Another aspect of the present invention pertains to use of an active compound, as described herein, in the manufacture of a medicament for use in treatment.

Another aspect of the present invention is a method of treatment, comprising administering to a patient in need of treatment a therapeutically effective amount of an active compound, as described herein.

In one embodiment, the treatment is treatment of a condition that encounters hypoxic conditions as it proceeds.

In one embodiment, the treatment is treatment of a condition that is characterised by inappropriate, excessive, and/or undesirable angiogenesis.

In one embodiment, the treatment is treatment of a condition characterised by hypoxia-induced angiogenesis.

In one embodiment, the treatment is treatment of angiogenesis in which the activity of HIF-1α is upregulated due to hypoxia.

In one embodiment, the treatment is treatment of a condition selected from: cancer, psoriasis, atherosclerosis, menorrhagia, endometrosis, arthritis (both inflammatory and rheumatoid), macular degeneration, Paget's disease, retinopathy and its vascular complications (including proliferative and diabetic retinopathy), benign vascular proliferation, fibroses, obesity and inflammation.

In one embodiment, the treatment is treatment of a proliferative condition.

In one embodiment, the treatment is treatment of cancer.

In one embodiment, the treatment is treatment of solid tumour cancer.

In one embodiment, the treatment is treatment of cancer selected from: phaeochromocytoma, paraganglioma, leiomyoma, renal cell carcinoma, gastric carcinoma, and colorectal carcinoma.

In one embodiment, the treatment is treatment of cancer (e.g., tumours) characterised by (e.g., that exhibits) SDH dysfunction.

In one embodiment, the treatment is treatment of cancer that develops SDH down-regulation in a later stage of the disease.

In one embodiment, the treatment is treatment of gastric or colorectal cancer, for example, Dukes' stage C of colorectal cancer.

In one embodiment, the treatment is treatment of oral carcinoma tumours.

In one embodiment, the treatment is treatment of cancer in which the activity of HIF-1α is upregulated due to hypoxia.

In one embodiment, the treatment is treatment of cancer in which the activity of one of the enzymes of the TCA cycle (e.g., succinate dehydrogenase, fumarate hydratase) is down-regulated.

In one embodiment, the patient being treated has inherited or somatic mutations in subunits A, B, C or D of the SDH gene or FH or down regulation of the expression of any of the SDH genes (subunits A, B, C or D) or of FH or impaired activity of the enzymes encoded by said genes.

Another aspect of the present invention is a method of treatment comprising co-administering to a patient in need of treatment; (a) a therapeutically effective amount of an active compound, as described herein, and (b) a second agent.

In one embodiment, the second agent is a compound that is an enhancer of aminolaevulinic acid (ALA) synthase.

In one embodiment, the second agent is selected from: barbiturates, anticonvulsants, non-narcotic analgetics, and non-steriodal anti-inflammatory compounds.

In one embodiment, the second agent is selected from: Allyl isopropyl acetamide, Phenobarbital, Deferoxamine, Felbamate, Lamotrigine, Tiagabine, Cyclophosphamide, N-methylprotoporphyrin, Succinyl-acetone, Carbamazepine, Ethanol, Phenyloin, Azapropazone, Chloroquine, Paracetamol, Griseofulvin, Cadmium, Iron, Pyridoxine.

In one embodiment, the second agent is selected from: Ethosuximide, Diazepam, Hydantoins, Methsuximide, Paramethadione, Phenobarbitone, Phensuximide, Phenyloin, Primidone, Succinimides, Bromides, Aspirin, Dihydroergotamine-Mesylate, Ergotamine Tartrate, Chloramphenicol, Dapsone, Erythromycin, Flucloxacillin, Pyrazinamide, Sulphonamides, Ampicillin, Vancomycin, Sulphonylureas Glipizidelnsulin, Alpha tocopheryl acetate, Ascorbic Acid, Folic Acid, Fructose, Glucose, Haem Arginate, Amidopyrine, Dichloralphenazone, Diclofenac Na, Dipyrone, Oxyphenbutazone, Propyphenazone, Aspitin, Codeine PO4, Dihydrocodeine, Canthaxanthin, β Carotene.

In one embodiment, the method further comprises the step of subjecting the patient to photodynamic therapy.

Another aspect of the present invention is a method of treatment comprising the steps of: (i) simultaneous, separate, or sequential administration of: (a) a first agent, that is an active compound, as described herein; and (b) a photosensitizer; followed by (ii) light irradiation.

Another aspect of the present invention pertains to a kit comprising (a) an active compound, as described herein, preferably provided as a pharmaceutical composition and in a suitable container and/or with suitable packaging; and (b) instructions for use, for example, written instructions on how to administer the active compound.

Another aspect of the present invention pertains to compounds obtainable by a method of synthesis as described herein, or a method comprising a method of synthesis as described herein.

Another aspect of the present invention pertains to compounds obtained by a method of synthesis as described herein, or a method comprising a method of synthesis as described herein.

Another aspect of the present invention pertains to novel intermediates, as described herein, which are suitable for use in the methods of synthesis described herein.

Another aspect of the present invention pertains to the use of such novel intermediates, as described herein, in the methods of synthesis described herein.

As will be appreciated by one of skill in the art, features and preferred embodiments of one aspect of the invention will also pertain to other aspect of the invention.

### BRIEF DESCRIPTION OF THE DRAWINGS

FIG. 1 shows:

(A) a photograph of an SDS-PAGE gel for cell extracts mixed with in vitro-translated HA-ODDD (HA-D) in the presence of Fe²⁺, ascorbate, and α-ketoglutarate, treated with: 0, 0.1, 0.5, 1.0, and 5.0 mM dimethyl ester succinic acid (DMS) (lanes 1-5) and with deferoxamine (DFO) (lane 6), showing HA-D and HA-D-OH;
(B) a photograph of a western blot gel for cell extracts: treated with CoCl₂ (left lane), untreated (2nd, 3rd, and 4th lanes), and treated with dimethyl ester succinic acid (DMS), for 48 hours under normoxic conditions, showing HIF-1α and actin.

Briefly, succinate inhibits PHD activity in vitro and induces HIF-1α levels in cells. Cell extracts were mixed with in vitro-translated HA-ODDD (HA-D) in the presence of Fe²⁺, ascorbate and α-ketoglutarate. Where indicated, succinate was added to the reaction. Deferoxamine (DFO), an iron chelator, was added to inhibit PHD activity. The hydroxylated polypeptide (HA-D-OH) migrates faster on SDS-PAGE. HIF-1α and actin levels were assessed by western blot of extracts from either untreated cells or from cells treated with dimethyl ester succinic acid (DMS) or CoCl₂ for 48 hours under normoxic conditions.

FIG. 2 shows:

(A) a photograph of a RT-PCR gel for extracts of cells (in triplicate) that were transfected with: scrambled siRNA (scRNAi), siRNA directed at SDHD subunit (Di3 or Di4)), showing SDHD and actin;
(B) a bar graph showing succinate-DCIP oxidoreductase (SDH) activity (nmol/min/1 mg) for the cells transfected as in (A);
(C) a photograph of a western blot gel for cell extracts of the cells transfected as in (A), showing HIF-1α and Actin; (D) a bar graph showing HIF transcriptional activity (as measured by the dual luciferase reporter assay system (Promega) using pGL2/HRE-Luciferase as a reporter for HIF activity, and recorded as HRE-luciferase intensity) (light units ×1000) following SDH inhibition for the cells transfected as in (A)

Briefly, inhibition of SDH activity increases HIF-1α levels and HIF activity. Cells were transfected (in triplicate) with either scrambled siRNA (scRNAi) or siRNA directed at SDHD subunit (Di3 or Di4). Following transfections, mRNA levels of SDHD and actin were quantified by RT-PCR. Succinate-DCIP oxidoreductase activity was analysed in cells transfected as in panel A, to confirm inhibition of SDH activity. HIF-1α levels were detected by western blot analysis following transfection with scRNAi, Di3 or Di4. Actin was used as loading control. HIF transcriptional activity following SDH inhibition was assessed by the dual luciferase reporter assay system (Promega) using pGL2/HRE-Luciferase as a reporter for HIF activity.

FIG. 3 shows:

(A) GCMS profiles of selected ionized fragments in extracts from cells transfected with scRNAi or Di3 (relative abundance (%) vs. retention time (minutes) vs. mass-to-charge ratio (amu); and
(B) a bar graph showing succinic acid levels (pmol/10⁶ cells) as determined using GCMS for cells transfected with: scrambled siRNA (scRNAi), siRNA directed at SDHD subunit (Di3 or Di4).

Briefly, SDH inhibition leads to increased levels of succinate. GCMS profiles of selected ionized fragments in extracts from cells transfected with either scRNAi or Di3 were recorded. Deuterated (D₄)-succinic acid was used as a reference and was identified by its major ion component of 119 amu at retention time of 8.05 min. Succinic acid was identified by its major ion component of 115 amu at retention time of 8.15 minutes. An increase in the succinic acid level is seen in Di3-transfected cells compared to scRNAi-transfected cells. Cells were transfected with the indicated siRNA construct and analyzed by GCMS as in panel A. Succinic acid levels were calculated as picomol per 10⁶ cells for each transfection and the results summarized from two independent experiments each done in triplicate.

FIG. 4 shows:

(A) photographs showing GFP fluorescence for each of:
(i) cells transfected with plasmids encoding GFP, without HA-pVHL, with scRNAi;
(ii) cells transfected with plasmids encoding GFP, with HA-pVHL, with scRNAi;
(iii) cells transfected with plasmids encoding GFP-ODDD, without HA-pVHL, with scRNAi;
(iv) cells transfected with plasmids encoding GFP-ODDD, with HA-pVHL, with scRNAi;
(v) cells transfected with plasmids encoding GFP-ODDD, with HA-pVHL, with Di3;
(vi) cells transfected with plasmids encoding GFP-ODDD, with HA-pVHL, with Di4.
(B) photographs of gels for extracts of cells (in triplicate) of (iv), (v), and (vi) in (A);
(C) a bar graph showing GFP fluorescence for extracts of cells of (i), (iii), and (v) in (A);
(D) photographs of far-western blot gels for cells transfected with plasmids encoding GFP-ODDD and scRNA, Di3, or Di4 (but without HA-pVHL), showing GFP-ODDD, GFP-ODDD/HA-pVHL, and HA-pVHL.

Briefly, inhibition of SDH decreases PHD activity. Cells were transfected with plasmids encoding either GFP (i and ii) or GFP-ODDD (iii, iv, v, vi), with (ii, iv, v, vi) or without (i, iii) HA-pVHL, and with one of the siRNA constructs: scRNAi (i, ii, iii, iv), Di3 (v) or Di4 (vi). GFP fluorescence was detected microscopically. GFP-ODDD and HA-pVHL protein levels were measured for cells transfected (in triplicate) as in panel A, iv, v, vi. Cells were transfected with plasmids encoding GFP or GFP-ODDD together with HA-pVHL and with the indicated siRNA. GFP fluorescence was analyzed in cell extracts before and after immunoprecipitation with an anti-HA antibody. The results are presented as the percent of GFP fluorescence bound to HA-pVHL and are the average and standard deviation of three independent transfections. Direct detection of ODDD hydroxylation was performed by far-western blot analysis. Cells were transfected (in triplicate) with plasmids encoding GFP-ODDD and the indicated siRNA (but without HA-pVHL). Protein extracts were blotted onto nitrocellulose membrane and the binding of immunopurified HA-pVHL to the blotted GFP-ODDD protein was detected using an anti-HA antibody. 10 ng of the immuno-purified HA-pVHL protein was loaded on lane 10.

FIG. 5 shows a schematic model that summarises the role of succinate in the mitochondrion-to-cytosol signalling pathway.

Briefly, succinate accumulated in the mitochondria due to SDH inhibition is transported to the cytosol. Elevated cytosolic succinate inhibits PHD and thereby HIFα hydroxylation. Consequently, pVHL binding to HIFα is decreased and elevated HIF activity induces expression of genes that facilitate angiogenesis, metastasis and glycolysis, leading to more aggressive tumours.

FIG. 6 shows:

(A) a photograph of an SDS-PAGE gel for cell extracts mixed with in vitro-translated ODD and treated with: 0, 0, 0, 1.0, 1.0, and 1.0 mM succinate (Succ) (lanes 1-6) and 0.01, 0.1, 1.0, 0.01, 0.1 and 1.0 mM free α-ketoglutaric acid (α-KG), showing ODD and OH-ODD;
(B) a bar graph showing intracellular α-ketoglutarate levels in cells treated with octyl-α-ketoglutarate (octyl-αKG), TFMB-α-ketoglutarate (TFMB α-KG), free α-ketoglutaric acid (αKG) or left untreated (control).

Briefly, succinate-mediated inhibition of PHD can be overcome by increasing α-ketoglutarate levels in vitro. A hydroxylation reaction of the ODD domain was carried out in vitro with the indicated amounts of succinate and α-ketoglutarate. Hydroxylation of ODD (OH-ODD) resulted in a faster migrating band on SDS-PAGE. Cells were either left untreated or treated for 5 hours with 1 mM of the indicated α-ketoglutarate ester or with free α-ketoglutaratic acid. The intracellular α-ketoglutarate level was analyzed using the glutamate dehydrogenase reaction.

FIG. 7 shows:

(A) a photograph of an SDS-PAGE gel showing levels of GFP-ODD fusion protein, GFP, HA-pVHL or actin (loading control) in extracts from control (Co) cells or clones (C2 and C3) co-expressing the GFP-ODD fusion protein and HA-tagged pVHL. Cells were either left untreated (U) or treated with CoCl₂ (CC);
(B) a photograph of an SDS-PAGE gel showing levels of GFP-ODD fusion protein and actin (loading control) in Clone 3 cells either left untreated (lanes 3-4), or treated with CoCl₂ (lanes 1-2) or dimethyl succinate (DMS) (lanes 5-10) for 48 hours. α-ketoglutarate esters (octyl-α-ketoglutarate (O) (lanes 7-8) or TFMB-α-ketoglutarate (O) (lanes 9-10) were added for the final 24 hours of the incubation (with DMS).

Briefly, the inhibition of PHD activity by succinate in cells is alleviated by the increase of intracellular α-ketoglutarate level. (A) Left panels—Clones (C2 and C3) co-expressing the GFP-ODD fusion protein and HA-tagged pVHL were analyzed by western blot. Cells transiently transfected with a plasmid encoding for GFP alone were used as a reference for GFP molecular weight (Co). Actin was used as a loading control. Right Panels—Clone 3 (C3) cells were either left untreated or treated with the hypoxia mimetic compound CoCl₂ and GFP-ODD and HA-pVHL protein levels were analyzed by western blot. (B) Clone 3 cells were treated as in Panel B and GFP-ODD levels were detected by western blot. CoCl₂-treated cells were used as a positive control for PHD inhibition and actin level was used as a loading control.

FIG. 8 shows:

(A) a photograph of a western blot for HEK293 cell extracts untreated (U; lanes 1-2) or treated with dimethyl ester succinic acid (DMS; lanes 3-8) and octyl-α-ketoglutarate (O; lanes 5-6) or TFMB-α-ketoglutarate (T; lanes 7-8) showing HIF-1α and actin;
(B) a bar-graph showing intracellular α-ketoglutarate levels (μM) in cells transfected with scrambled siRNA (Sc) or siRNA directed at SDH-D subunit (Di3) and treated with or without octyl-α-ketoglutarate (octyl-α-KG);
(C) a photograph of a western blot for cell extracts transfected as in (B) and treated with TFMB-α-ketoglutarate (T; lanes 5-6) or octyl-α-ketoglutarate (O; lanes 7-8) showing HIF-1α and actin.

Briefly, α-ketoglutarate re-targets HIF1α for degradation. HEK293 cells were either untreated or treated with DMS and/or the indicated α-ketoglutarate ester and HIF1α protein level was detected by western blot. Actin was used as loading control. Cells were transfected with either the control scrambled shRNA (Sc) or shRNA targeting SDHD (Di3) and 36 hours later were either left untreated or treated with 1 mM octyl-α-ketoglutarate for 5 hours. The intracellular level of α-ketoglutarate was analyzed as described. Cells were treated with ascorbate and N-acetyl cysteine and transfected with either scrambled control shRNA (Sc) or shRNA targeting SDHD (Di3). Where indicated, the different α-ketoglutarate esters were added and HIF1α protein level was detected by western blot.

FIG. 9 shows a bar-graph showing cell viability (% of untreated) of cells grown in the presence or absence of an SDH inhibitor (TTFA) and treated with control (DMSO), octyl-α-ketoglutarate (Octyl αKG), TFMB-α-ketoglutarate (TFMB αKG) or free α-ketoglutaric acid (αKG).

Briefly, cells were grown continuously in the presence or absence of a succinate dehydrogenase (SDH) inhibitor—(TTFA) in a medium that can sustain cells with dysfunctional oxidative phosphorylation (with excess of pyruvate and uridine). Where indicated, cells were either treated with vehicle control (DMSO), free α-ketoglutaric acid (αKG) as control or with TFMB-α-ketoglutarate (T-αKG) or with octyl-α-ketoglutarate (O-αKG). Only the treatment that lead to increase intracellular α-ketoglutarate (T-αKG or O-αKG) in cells with dysfunctional SDH activity lead to a significant death. Viability was quantified using an MTT assay (Roche).

FIG. 10 shows photographs of western blot gels for cell extracts of cells incubated for 3 hours under low oxygen (3%) (conditions sufficient for HIF-1α induction in these cells) in the presence of DMSO (D) as vehicle control; α-ketoglutaric acid benzyl ester (B) (4 mM); and α-ketoglutaric acid trifluoromethylbenzyl ester (T) (4 mM), showing HIF-1α and actin.

Briefly, α-ketoglutarate blocks HIF-1α induction under hypoxic conditions. HEK293 cells were incubated for 3 hours under low oxygen (3%) (conditions sufficient for HIF-1α induction in these cells) in the presence of the following compounds: D=DMSO vehicle control; B=α-ketoglutaric acid benzyl ester (4 mM); T=α-ketoglutaric acid trifluoromethylbenzyl ester (4 mM). Cells were lysed and extracts were analysed by western blot using anti-HIF-1α or anti-actin antibodies.

### DETAILED DESCRIPTION OF THE INVENTION

The inventors have determined that, surprisingly and unexpectedly, α-ketoglutarate significantly enhances PHD activity in cells under low oxygen (e.g., hypoxic) conditions.

The inventors have also determined that, surprisingly and unexpectedly, succinate, which accumulates in cells as a result inhibition of the tricarboxylic acid (TCA) cycle in the mitochondria, inhibits HIFα prolyl hydroxylases in the cytosol, leading to stabilization and activation of HIF-1α in cells in which the activity of one of the enzymes of the TCA cycle is down regulated. The inventors have also determined that this stabilization and activation of HIF-1α can be overcome by supplying α-ketoglutarate (e.g., via an α-ketoglutarate ester) to the cell.

The inventors have identified a new signalling pathway that connects mitochondrial metabolic dysfunction with cancer. For the first time, a mechanism that links down-regulation of SDH to HIF-1α induction has been elucidated. The inventors have shown that accumulation of succinate, due to SDH inhibition, transmits an “oncogenic” signal from the mitochondria to the cytosol. Once in the cytosol, succinate inhibits HIFα prolyl hydroxylase and leads to HIF-1α stabilization in the presence of wild type pVHL. Thus, succinate can modulate nuclear events by inducing HIF transcriptional activity, hence increasing expression of genes that facilitate angiogenesis, metastasis, and glycolysis, ultimately leading to tumour progression. This mitochondrion-to-cytosol pathway identifies succinic acid, for the first time, as an intracellular messenger. See FIG. 5.

The inventors have developed methods for treating hypoxia-induced angiogenesis, including, for example, methods of treating or preventing cancer in which the activity of one of the enzymes of the TCA cycle is down regulated, by specifically activating HIFα hydroxylases, preferably HIFα prolyl hydroxylases.

The inventors have also identified a class of compounds that are useful in methods for the treatment of hypoxia-induced angiogenesis, including, for example, methods for the treatment and prevention of cancer.

These compounds establish normal HIF-1α levels by activating HIFα hydroxylase, Consequently, these compounds can be used to restore normal HIFα levels under hypoxic conditions. Furthermore, these compounds can be used in the treatment of tumours in which the activity of one of the enzymes of the TCA cycle is down regulated. For example, these compounds can be used to overcome the inhibitory effect of succinate on HIF prolyl α-hydroxylase, thereby restoring normal HIF-1α levels in tumours in which the activity of one of the enzymes of the TCA cycle is down regulated. Establishing normal levels of HIF-1α has a profound effect on the vascularization of the tumour as well as its ability to metastasise, which are two major oncogenic processes that are induced by HIF activity.

### The Compounds

One aspect of the invention pertains to certain compounds (referred to herein as “α-ketoglutarate compounds” or “α-ketogluartates” or “α-ketogluartate esters”), for example, that activate HIFα hydroxylase. These compounds may conveniently be described as α-ketoglutarates bearing (e.g., conjugated to, coupled to) a hydrophobic moiety.

For example, these compounds may be described as α-ketoglutarate esters (i.e., esters of α-ketogluartic acid) having a hydrophobic moiety that is, or is part of, an ester group (i.e., —C(═O)OR) formed from one of the acid groups of α-ketogluartic acid.

For reference, the related parent compounds, glutaric acid and α-ketoglutaric acid are shown below.

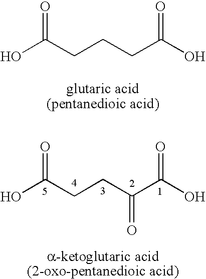

Thus, in one embodiment, the compounds have the following formula:

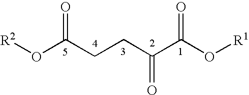

wherein each of R¹ and R² is independently selected from:
(i) H; and
(ii) a hydrophobic moiety;
with the proviso that R¹ and R² are not both H;
   and pharmaceutically acceptable salts, solvates, amides, esters, ethers, N-oxides, chemically protected forms, and prodrugs thereof.

### The Groups R¹ and R²

In one embodiment, neither R¹ nor R² is H (i.e., diesters).

In one embodiment, neither R¹ nor R² is H; and R¹ and R² are different.

In one embodiment, neither R¹ nor R² is H; and R¹ and R² are identical.

In one embodiment, exactly one of R¹ and R² is H (i.e., monoesters).

In one embodiment, R¹ is H (and R² is not H):

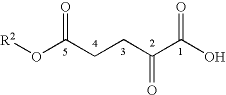

In one embodiment, R² is H (and R² is not H):

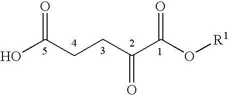

### The Hydrophobic Moiety/Moieties

As used herein, the term “hydrophobic moiety” includes, but is not limited to, chemical moieties with non-polar atoms or groups that have a tendency to interact with each other rather than with water or other polar atoms or groups. Hydrophobic moieties are substantially insoluble or only poorly soluble in water.

Optionally, the hydrophobic moiety may be selected according to their fusogenic properties or their interactions with components of cellular membranes, such as lectins and lipid head groups. For example, the hydrophobic moiety may comprise a polymer (e.g., a linear or branched polymer); an alkyl, alkenyl, and/or alkynyl group, which may be, for example, linear, branched or cyclic (e.g., C₁-C₃₀ alkyl, C₂-C₃₀ alkenyl, C₂-C₃₀ alkynyl, C₃-C₃₀ cycloalkyl, C₃-C₃₀ cylcoalkenyl, C₃-C₃₀ cycloalkynyl); an aromatic group (e.g., C₆-C₂₀ carboaryl, C₅-C₂₀ heteroaryl); or a combination thereof.

Optionally, the hydrophobic moiety may comprise one or more of: a heteroatom, a heterocyclic group, a peptide, a peptoid, a natural product, a synthetic compound, a steroid, and a steroid derivative (e.g., hydrophobic moieties which comprise a steroidal nucleus, e.g., a cholesterol ring system).

It is intended that the hydrophobic moiety be selected so that the α-ketoglutarate compound is capable of performing its intended function, e.g., to cross through lipid membranes into the cytosol/mitochondria.

Examples of hydrophobic moieties include, but are not limited to, those derived from: lipids, fatty acids, phospholipids, sphingolipids, acylglycerols, waxes, sterols, steroids (e.g., cholesterol), terpenes, prostaglandins, thromboxanes, leukotrienes, isoprenoids, retenoids, biotin, and hydrophobic amino acids (e.g., tryptophan, phenylalanine, isoleucine, leucine, valine, methionine, alanine, proline, and tyrosine).

In one embodiment, the hydrophobic moiety, or each hydrophobic moiety, is independently selected from:
C₁-C₃₀ alkyl;
C₂-C₃₀ alkenyl;
C₂-C₃₀ alkynyl;
C₃-C₃₀ cycloalkyl;
C₃-C₃₀ cycloalkenyl;
C₃-C₃₀ cycloalkynyl;
C₆-C₂₀ carboaryl;
C₅-C₂₀ heteroaryl;
C₆-C₂₀ carboaryl-C₁-C₇ alkyl;
C₅-C₂₀ heteroaryl-C₁-C₇ alkyl;
and is unsubstituted or substituted.

In one embodiment, the hydrophobic moiety, or each hydrophobic moiety, is independently selected from:
C₁-C₃₀ alkyl;
C₂-C₃₀ alkenyl;
C₂-C₃₀ alkynyl;
and is unsubstituted or substituted.

In one embodiment, the bottom of the range (for alkyl, alkenyl, alkynl) is C₄.

In one embodiment, the bottom of the range is C₆.

In one embodiment, the bottom of the range is C₈.

In one embodiment, the bottom of the range is C₁₀.

In one embodiment, the bottom of the range is C₁₂.

In one embodiment, the top of the range (for alkyl, alkenyl, alkynl) is C₃₀.

In one embodiment, the top of the range is C₂₄.

In one embodiment, the top of the range is C₂₂.

In one embodiment, the top of the range is C₂₀.

In one embodiment, the top of the range is C₁₈.

In one embodiment, the top of the range is C₁₆.

In one embodiment, the range (for alkyl, alkenyl, alkynl) is C₄-C₂₀.

In one embodiment, the range is C₆-C₁₈.

In one embodiment, the range is C₈-C₁₁.

In one embodiment, the range is C₁₀-C₂₄.

In one embodiment, the range is C₁₂-C₂₂.

In one embodiment, the range is C₁₄-C₂₀.

In one embodiment, the range is C₁₆-C₁₈.

In one embodiment, the hydrophobic moiety, or each hydrophobic moiety, is independently C₁-C₃₀ alkyl and is unsubstituted or substituted.

In one embodiment, the bottom of the range (for alkyl) is C₄.

In one embodiment, the bottom of the range is C₆.

In one embodiment, the bottom of the range is C₈.

In one embodiment, the bottom of the range is C₁₀.

In one embodiment, the bottom of the range is C₁₂.

In one embodiment, the top of the range (for alkyl) is C₃₀.

In one embodiment, the top of the range is C₂₄.

In one embodiment, the top of the range is C₂₂.

In one embodiment, the top of the range is C₂₀.

In one embodiment, the top of the range is C₁₁.

In one embodiment, the top of the range is C₁₆.

In one embodiment, the range (for alkyl) is C₄-C₂₀.

In one embodiment, the range is C₆-C₁₈.

In one embodiment, the range is C₈-C₁₆.

In one embodiment, the range is C₁₀-C₂₄.

In one embodiment, the range is C₁₂-C₂₂.

In one embodiment, the range is C₁₄-C₂₀.

In one embodiment, the range is C₁₆-C₁₈.

In one embodiment, the alkyl group is a linear or branched alkyl group and is unsubstituted or substituted, for example, in one embodiment, the hydrophobic moiety is linear or branched C₁-C₃₀ alkyl and is unsubstituted or substituted.

In one embodiment, the hydrophobic moiety, or each hydrophobic moiety, is independently —(CH₂)ₙCH₃, wherein n is independently an integer from 0 to 29.

In one embodiment, the bottom of the range for n is 3.

In one embodiment, the bottom of the range for n is 5.

In one embodiment, the bottom of the range for n is 7.

In one embodiment, the bottom of the range for n is 9.

In one embodiment, the bottom of the range for n is 11.

In one embodiment, the top of the range for n is 29.

In one embodiment, the top of the range for n is 23.

In one embodiment, the top of the range for n is 21.

In one embodiment, the top of the range for n is 19.

In one embodiment, the top of the range for n is 17.

In one embodiment, the top of the range for n is 15.

In one embodiment, n is independently an integer from 3 to 19.

In one embodiment, n is independently an integer from 5 to 17.

In one embodiment, n is independently an integer from 7 to 15.

In one embodiment, the hydrophobic moiety, or each hydrophobic moiety, is independently selected from:
C₆-C₂₀ carboaryl;
C₅-C₂₀ heteroaryl;
C₆-C₂₀ carboaryl-C₁-C₇ alkyl;
C₅-C₂₀ heteroaryl-C₁-C₇ alkyl;
and is unsubstituted or substituted.

In one embodiment, the hydrophobic moiety, or each hydrophobic moiety, is independently selected from:
C₆-C₁₂ carboaryl;
C₅-C₁₂ heteroaryl;
C₆-C₁₂ carboaryl-C₁-C₇ alkyl;
C₅-C₁₂ heteroaryl-C₁-C₇ alkyl;
and is unsubstituted or substituted.

In one embodiment, the hydrophobic moiety, or each hydrophobic moiety, is independently selected from:
C₆-C₁₀ carboaryl;
C₅-C₁₀ heteroaryl;
C₆-C₁₀ carboaryl-C₁-C₇ alkyl;
C₅-C₁₀ heteroaryl-C₁-C₇ alkyl;
and is unsubstituted or substituted.

In one embodiment, the hydrophobic moiety, or each hydrophobic moiety, is independently selected from:
C₆-C₂₀ carboaryl;
C₆-C₂₀ carboaryl-C₁-C₇ alkyl;
and is unsubstituted or substituted.

In one embodiment, the hydrophobic moiety, or each hydrophobic moiety, is independently selected from:
C₆-C₁₂ carboaryl;
C₆-C₁₂ carboaryl-C₁-C₇ alkyl;
and is unsubstituted or substituted.

In regard to the phrase “unsubstituted or substituted”, any substituents, if present, may be, in one embodiment, as defined below for R^{P}.

For example, in one embodiment, each carboaryl and heteroaryl group, if present, is unsubstituted or substituted with one or more (e.g., 1, 2, 3, 4, etc.) substituents independently selected from: halo; cyano; nitro; hydroxy; C₁-C₇ alkyoxy; C₁-C₇ alkyl; C₁-C₇ haloalkyl; and C₈-C₃₀ alkyl.

In one embodiment, the above C₈-C₃₀ alkyl groups are C₁₀-C₂₄ alkyl.

In one embodiment, the above C₈-C₃₀ alkyl groups are C₁₂-C₂₂ alkyl.

In one embodiment, the above C₈-C₃₀alkyl groups are C₁₄-C₂₀ alkyl.

In one embodiment, the above C₈-C₃₀ alkyl groups are C₁₆-C₁₈ alkyl.

In one embodiment, the hydrophobic moiety, or each hydrophobic moiety, is independently an optionally substituted phenyl group of formula:

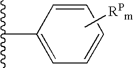

wherein m is independently 0, 1, 2, 3, 4, or 5, and each R^{P}, if present, is independently a substituent.

In one embodiment, the hydrophobic moiety, or each hydrophobic moiety, is independently an optionally substituted benzyl group of formula:

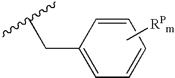

wherein m is independently 0, 1, 2, 3, 4, or 5, and each R^{P}, if present, is independently a substituent.

In one embodiment, m is 0, 1, 2, or 3.

In one embodiment, m is 0, 1, or 2.

In one embodiment, m is 0 or 1.

In one embodiment, the substituents, R^{P}, are independently selected from the following:

(1) carboxylic acid; (2) ester; (3) amido or thioamido; (4) acyl; (5) halo; (6) cyano; (7) nitro; (8) hydroxy; (9) ether; (10) thiol; (11) thioether; (12) acyloxy; (13) carbamate; (14) amino; (15) acylamino or thioacylamino; (16) aminoacylamino or aminothioacylamino; (17) sulfonamino; (18) sulfonyl; (19) sulfonate; (20) sulfonamido; (21) C₅₋₂₀aryl-C₁₋₇alkyl; (22) C₆₋₂₀-carboaryl and C₅₋₂₀heteroaryl; (23) C₃₋₂₀heterocyclyl; (24) C₁₋₇alkyl; C₈₋₃₀alkyl; C₂₋₇alkenyl; C₂₋₇alkynyl; C₃₋₇cycloalkyl; C₃₋₇cycloalkenyl; C₃₋₇cycloalkynyl.

In one embodiment, the substituents, R^{P}, are independently selected from the following:
(1) —C(═O)OH;
(2) —C(═O)OR¹, wherein R¹ is independently as defined in (21), (22), (23) or (24);
(3) —C(═O)NR²R³ or —C(═S)NR²R³, wherein each of R² and R³ is independently —H; or as defined in (21), (22), (23) or (24); or R² and R³ taken together with the nitrogen atom to which they are attached form a ring having from 3 to 7 ring atoms;
(4) —C(═O)R⁴, wherein R⁴ is independently —H, or as defined in (21), (22), (23) or (24);
(5) —F, —Cl, —Br, —I;
(6) —CN;
(7) —NO₂;
(8) —OH;
(9) —OR⁵, wherein R⁵ is independently as defined in (21), (22), (23) or (24);
(10) —SH;
(11) —SR⁶, wherein R⁶ is independently as defined in (21), (22), (23) or (24);
(12) —OC(═O)R⁷, wherein R⁷ is independently as defined in (21), (22), (23) or (24);
(13) —OC(═O)NR⁸R⁹, wherein each of R⁸ and R⁹ is independently —H; or as defined in (21), (22), (23) or (24); or R⁸ and R⁹ taken together with the nitrogen atom to which they are attached form a ring having from 3 to 7 ring atoms;
(14) —NR¹⁰R¹¹, wherein each of R¹⁰ and R¹¹ is independently —H; or as defined in (21), (22), (23) or (24); or R¹⁰ and R¹¹ taken together with the nitrogen atom to which they are attached form a ring having from 3 to 7 ring atoms;
(15) —NR¹²C(═O)R¹³ or —NR¹²C(═S)R¹³, wherein R¹² is independently —H; or as defined in (21), (22), (23) or (24); and R¹³ is independently —H, or as defined in (21), (22), (23) or (24);
(16) —NR¹⁴C(═O)NR¹⁵R¹⁶ or —NR¹⁴C(═S)NR¹⁵R¹⁶, wherein R¹⁴ is independently —H; or as defined in (21), (22), (23) or (24); and each of R¹⁵ and R¹⁶ is independently —H; or as defined in (21), (22), (23) or (24); or R¹⁵ and R¹⁶ taken together with the nitrogen atom to which they are attached form a ring having from 3 to 7 ring atoms;
(17) —NR¹⁷SO₂R¹⁸, wherein R¹⁷ is independently —H; or as defined in (21), (22), (23) or (24); and R¹⁸ is independently —H, or as defined in (21), (22), (23) or (24);
(18) —SO₂R¹⁹, wherein R¹⁹ is independently as defined in (21), (22), (23) or (24);
(19) —OSO₂R²⁰ and wherein R²⁰ is independently as defined in (21), (22), (23) or (24);
(20) —SO₂NR²¹R²², wherein each of R²¹ and R²² is independently —H; or as defined in (21), (22), (23) or (24); or R²¹ and R²² taken together with the nitrogen atom to which they are attached form a ring having from 3 to 7 ring atoms;
(21) C₅₋₂₀aryl-C₁₋₇alkyl, for example, wherein C₅₋₂₀aryl is as defined in (22); unsubstituted or substituted, e.g., with one or more groups as defined in (1) to (24);
(22) C₆₋₂₀carboaryl; C₅₋₂₀heteroaryl; unsubstituted or substituted, e.g., with one or more groups as defined in (1) to (24);
(23) C₃₋₂₀heterocyclyl; unsubstituted or substituted, e.g., with one or more groups as defined in (1) to (24);
(24) C₁₋₇alkyl; C₈₋₃₀alkyl; C₂₋₇alkenyl; C₂₋₇alkynyl; C₃₋₇cycloalkyl; C₃₋₇cycloalkenyl; C₃₋₇cycloalkynyl; unsubstituted or substituted, e.g., with one or more groups as defined in (1) to (23),
   e.g., halo-C₁₋₇alkyl;
   e.g., amino-C₁₋₇alkyl (e.g., —(CH₂)_{w}-amino, w is 1, 2, 3, or 4);
   e.g., carboxy-C₁₋₇alkyl (e.g., —(CH₂)_{w}—COOH, w is 1, 2, 3, or 4);
   e.g., acyl-C₁₋₇alkyl (e.g., —(CH₂)_{w}—C(═O)R⁴, w is 1, 2, 3, or 4);
   e.g., hydroxy-C₁₋₇alkyl (e.g., —(CH₂)_{w}—OH, w is 1, 2, 3, or 4);
   e.g., C₁₋₇alkoxy-C₁₋₇alkyl (e.g., —(CH₂)_{w}—O—C₁₋₇alkyl, w is 1, 2, 3, or 4).

In one embodiment, the substituents, R^{P}, are independently selected from the following:
(1) —C(═O)OH;
(2) —C(═O)OMe, —C(═O)OEt, —C(═O)O(iPr), —C(═O)O(tBu); —C(═O)O(cPr);
   —C(═O)OCH₂CH₂OH, —C(═O)OCH₂CH₂OMe, —C(═O)OCH₂CH₂OEt;
   —C(═O)OPh, —C(═O)OCH₂Ph;
(3) —(C═O)NH₂, —(C═O)NMe₂, —(C═O)NEt₂, —(C═O)N(iPr)₂, —(C═O)N(CH₂CH₂OH)₂;
   —(C═O)-morpholino, —(C═O)NHPh, —(C═O)NHCH₂Ph;
(4) —C(═O)H, —(C═O)Me, —(C═O)Et, —(C═O)(tBu), —(C═O)-cHex, —(C═O)Ph; —(C═O)CH₂Ph;
(5) —F, —Cl, —Br, —I;
(6) —CN;
(7) —NO₂;
(8) —OH;
(9) —OMe, —OEt, —O(iPr), —O(tBu), —OPh, —OCH₂Ph;
   —OCF₃, —OCH₂CF₃;
   —OCH₂CH₂OH, —OCH₂CH₂OMe, —OCH₂CH₂OEt;
   —OCH₂CH₂NH₂, —OCH₂CH₂NMe₂, —OCH₂CH₂N(iPr)₂;
   —OPh-Me, —OPh-OH, —OPh-OMe, —OPh-F, —OPh-Cl, —OPh-Br, —OPh-I;
(10) —SH;
(11) —SMe, —SEt, —SPh, —SCH₂Ph;
(12) —OC(═O)Me, —OC(═O)Et, —OC(═O)(iPr), —OC(═O)(tBu); —OC(═O)(cPr);
   —OC(═O)CH₂CH₂OH, —OC(═O)CH₂CH₂OMe, —OC(═O)CH₂CH₂OEt;
   —OC(═O)Ph, —OC(═O)CH₂Ph;
(13) —OC(═O)NH₂, —OC(═O)NHMe, —OC(═O)NMe₂, —OC(═O)NHEt, —OC(═O)NEt₂, —OC(═O)NHPh, —OC(═O)NCH₂Ph;
(14) —NH₂, —NHMe, —NHEt, —NH(iPr), —NMe₂, —NEt₂, —N(iPr)₂, —N(CH₂CH₂OH)₂;
   —NHPh, —NHCH₂Ph; piperidino, piperazino, morpholino;
(15) —NH(C═O)Me, —NH(C═O)Et, —NH(C═O)nPr, —NH(C═O)Ph, —NHC(═O)CH₂Ph;
   —NMe(C═O)Me, —NMe(C═O) Et, —NMe(C═O)Ph, —NMeC(═O)CH₂Ph;
(16) —NH(C═O)NH₂, —NH(C═O)NHMe, —NH(C═O)NHEt, —NH(C═O)NPh, —NH(C═O)NHCH₂Ph; —NH(C═S)NH₂, —NH(C═S)NHMe, —NH(C═S)NHEt, —NH(C═S)NPh, —NH(C═S)N HCH₂Ph;
(17) —NHSO₂Me, —NHSO₂Et, —NHSO₂Ph, —NHSO₂PhMe, —NHSO₂CH₂Ph;
   —NMeSO₂Me, —NMeSO₂Et, —NMeSO₂Ph, —NMeSO₂PhMe, —NMeSO₂CH₂Ph;
(18) —SO₂Me, —SO₂CF₃, —SO₂Et, —SO₂Ph, —SO₂PhMe, —SO₂CH₂Ph;
(19) —OSO₂Me, —OSO₂CF₃, —OSO₂Et, —OSO₂Ph, —OSO₂PhMe, —OSO₂CH₂Ph;
(20) —SO₂NH₂, —SO₂NHMe, —SO₂NHEt, —SO₂NMe₂, —SO₂NEt₂, —SO₂-morpholino, —SO₂NHPh, —SO₂NHCH₂Ph;
(21) —CH₂Ph, —CH₂Ph-Me, —CH₂Ph-OH, —CH₂Ph-F, —CH₂Ph-Cl;
(22) -Ph, -Ph-Me, -Ph-OH, -Ph-OMe, -Ph-NH₂, -Ph-F, -Ph-Cl, -Ph-Br, -Ph-I;
   pyridyl, pyrazinyl, pyrimidinyl, pyridazinyl; furanyl, thiophenyl, pyrrolyl, imidazolyl, pyrazolyl, oxazolyl, thiazolyl, thiadiazolyl;
(23) pyrrolidinyl, imidazolidinyl, pyrazolidinyl, piperidinyl, piperazinyl, azepinyl, tetrahydrofuranyl, tetrahydropyranyl, morpholinyl, azetidinyl;
(24) -Me, -Et, -nPr, -iPr, -nBu, -iBu, -sBu, -tBu, -nPe, -nHex; —(CH₂)₇CH₃, —(CH₂)_{g}CH₃,
   —(CH₂)₁₁CH₃, —(CH₂)₁₃CH₃, —(CH₂)₁₅CH₃, —(CH₂)₁₇CH₃, —(CH₂)₁₉CH₃;
   -cPr, -cHex; —CH═CH₂, —CH₂—CH═CH₂;
   —CF₃, —CHF₂, —CH₂F, —CCl₃, —CBr₃, —CH₂CH₂F, —CH₂CHF₂, and —CH₂CF₃;
   —CH₂OH, —CH₂OMe, —CH₂OEt, —CH₂NH₂, —CH₂NMe₂;
   —CH₂CH₂OH, —CH₂CH₂OMe, —CH₂CH₂OEt, —CH₂CH₂CH₂NH₂, —CH₂CH₂NMe₂.

In one embodiment, the substituents, R^{P}, are independently selected from: halo; cyano; nitro; hydroxy; C₁-C₇ alkyoxy; C₁-C₇ alkyl; C₁-C₇ haloalkyl; and C₆-C₃₀ alkyl.

In one embodiment, the substituents, R^{P}, are independently selected from: halo; cyano; nitro; hydroxy; C₁-C₄ alkyoxy; C₁-C₄ alkyl; C₁-C₄ haloalkyl; and C₁₂-C₂₂ alkyl.

In one embodiment, the substituents, R^{P}, are independently selected from: halo; C₁-C₄ alkyl; and C₁-C₄ haloalkyl.

In one embodiment, the substituents, R^{P}, are independently selected from: fluoro; C₁-C₄ alkyl; and C₁-C₄ fluoroalkyl.

In one embodiment, the substituents, R^{P}, are independently selected from: F, —CH₃, —CF₃.

As used herein, the term “halo” includes fluoro, chloro, bromo and iodo.

As used herein, the term “alkyl” pertains to monovalent, monodentate, aliphatic (linear or branched) saturated hydrocarbon moieties, for example, methyl, ethyl, n-propyl, i-propyl, etc.

Examples of (unsubstituted) alkyl groups include methyl (C₁), ethyl (C₂), propyl (C₃), butyl (C₄), pentyl (C₅), hexyl (C₆), heptyl (C₇), octyl (C₈), nonyl (C₉), decyl (C₁₀), undecyl (C₁₁), dodecyl (C₁₂), tridecyl (C₁₃), tetradecyl (C₁₄), pentadecyl (C₁₅), and eicodecyl (C₂₀).

Examples of (unsubstituted) linear alkyl groups include methyl (C₁), ethyl (C₂), n-propyl (C₃), n-butyl (C₄), n-pentyl (amyl) (C₅), n-hexyl (C₈), and n-heptyl (C₇).

Examples of (unsubstituted) branched alkyl groups include iso-propyl (C₃), iso-butyl (C₄), sec-butyl (C₄), tert-butyl (C₄), iso-pentyl (C₅), and neo-pentyl (C₅).

As used herein, the term “alkenyl” pertains to monovalent, monodentate, aliphatic (linear or branched) hydrocarbon moieties having at least one carbon-carbon double bond.

Examples of (unsubstituted) alkenyl groups include ethenyl (vinyl, —CH═CH₂), 1-propenyl (—CH═CH—CH₃), 2-propenyl (allyl, —CH—CH═CH₂), isopropenyl (1-methylvinyl, —C(CH₃)═CH₂), butenyl (C₄), pentenyl (C₅), and hexenyl (C₆).

As used herein, the term “alkynyl” pertains to monovalent, monodentate, aliphatic (linear or branched) hydrocarbon moieties having at least one carbon-carbon triple bond.

Examples of (unsubstituted) alkynyl groups include ethynyl (ethinyl, —C≡CH) and 2-propynyl (propargyl, —CH₂—C≡CH).

As used herein, the term “cycloalkyl” pertains to monovalent, monodentate, non-aromatic saturated hydrocarbon moieties having at least one carbon-atom ring (preferably having from 3 to 7 ring carbon atoms).

Examples of cycloalkyl groups include those derived from saturated monocyclic hydrocarbon compounds: cyclopropane (C₃), cyclobutane (C₄), cyclopentane (C₅), cyclohexane (C₆), cycloheptane (C₇), methylcyclopropane (C₄), dimethylcyclopropane (C₅), methylcyclobutane (C₅), dimethylcyclobutane (C₆), methylcyclopentane (C₆), dimethylcyclopentane (C₇), methylcyclohexane (C₇), dimethylcyclohexane (C₈), menthane (C₁₀); and saturated polycyclic hydrocarbon compounds: thujane (C₁₀), carane (C₁₀), pinane (C₁₀), bornane (C₁₀), norcarane (C₇), norpinane (C₇), norbornane (C₇), adamantane (C₁₀), decalin (decahydronaphthalene) (C₁₀).

As used herein, the term “cycloalkenyl” pertains to monovalent, monodentate, non-aromatic hydrocarbon moieties having at least one carbon-atom ring (preferably having from 3 to 7 ring carbon atoms) and at least one carbon-carbon double bond.

Examples of cycloalkenyl groups include those derived from unsaturated monocyclic hydrocarbon compounds: cyclopropene (C₃), cyclobutene (C₄), cyclopentene (C₅), cyclohexene (C₆), methylcyclopropene (C₄), dimethylcyclopropene (C₅), methylcyclobutene (C₅), dimethylcyclobutene (C₆), methylcyclopentene (C₆), dimethylcyclopentene (C₇), methylcyclohexene (C₇), dimethylcyclohexene (C₈); and unsaturated polycyclic hydrocarbon compounds: camphene (C₁₀), limonene (C₁₀), pinene (C₁₀).

As used herein, the term “cycloalkynyl” pertains to monovalent, monodentate, non-aromatic hydrocarbon moieties having at least one carbon-atom ring (preferably having from 3 to 7 ring carbon atoms) and at least one carbon-carbon triple bond.

As used herein, the term “aryl” pertains to monovalent, monodentate, moieties that have an aromatic ring and which has from 3 to 20 ring atoms (unless otherwise specified). Preferably, each ring has from 5 to 7 ring atoms. The ring atoms may be all carbon atoms, as in “carboaryl” groups or the ring atoms may include one or more heteroatoms (e.g., 1, 2, 3, 4, etc.) (e.g., selected from N, O, and S), as in “heteroaryl” groups. In this context, the prefixes (e.g., C₅-C₂₀, C₅-C₁₂, C₅-C₁₀, etc.) denote the number of ring atoms, or range of number of ring atoms, whether carbon atoms or heteroatoms.

Examples of carboaryl groups include those derived from benzene (i.e., phenyl) (C₆), naphthalene (C₁₀), azulene (C₁₀), anthracene (C₁₄), phenanthrene (C₁₄), naphthacene (C₁₈), and pyrene (C₁₆).

Examples of carboaryl groups which comprise fused rings, at least one of which is an aromatic ring, include groups derived from indane (e.g., 2,3-dihydro-1H-indene) (C₉), indene (C₉), isoindene (C₉), tetraline (1,2,3,4-tetrahydronaphthalene (C₁₀), acenaphthene (C₁₂), fluorene (C₁₃), phenalene (C₁₃), acephenanthrene (C₁₅), and aceanthrene (C₁₆).

Additional examples of carboaryl groups include groups derived from: indene (C₉), indane (e.g., 2,3-dihydro-1H-indene) (C₉), tetraline (1,2,3,4-tetrahydronaphthalene) (C₁₁), acenaphthene (C₁₂), fluorene (C₁₃), phenalene (C₁₃), acephenanthrene (C₁₅), aceanthrene (C₁₆), cholanthrene (C₂₀).

Examples of monocyclic heteroaryl groups include those derived from:

N₁: pyrrole (azole) (C₅), pyridine (azine) (C₆);
O₁: furan (oxole) (C₅);
S₁: thiophene (thiole) (C₅);
N₂O₁: oxazole (C₅), isoxazole (C₅), isoxazine (C₆);
N₂O₁: oxadiazole (furazan) (C₅);
N₃O₁: oxatriazole (C₅);
N₁S₁: thiazole (C₅), isothiazole (C₅);
N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆);
N₃: triazole (C₅), triazine (C₆); and,
N₄: tetrazole (C₅).

Examples of Polycyclic Heteroaryl Groups Include:

C₉heterocyclic groups (with 2 fused rings) derived from benzofuran (O₁), isobenzofuran (O₁), indole (N₁), isoindole (N₁), indolizine (N₁), indoline (N₁), isoindoline (N₁), purine (N₄) (e.g., adenine, guanine), benzimidazole (N₂), indazole (N₂), benzoxazole (NO₁), benzisoxazole (N₁O₁), benzodioxole (O₂), benzofurazan (N₂O₁), benzotriazole (N₃), benzothiofuran (S₁), benzothiazole (N₁S₁), benzothiadiazole (N₂S);
C₁₀heterocyclic groups (with 2 fused rings) derived from chromene (O₁), isochromene (O₁), chroman (O₁), isochroman (O₁), benzodioxan (O₂), quinoline (N₁), isoquinoline (N₁), quinolizine (N₁), benzoxazine (N₁O₁), benzodiazine (N₂), pyridopyridine (N₂), quinoxaline (N₂), quinazoline (N₂), cinnoline (N₂), phthalazine (N2), naphthyridine (N₂), pteridine (N₄);
C₁₁heterocylic groups (with 2 fused rings) derived from benzodiazepine (N₂);
C₁₃heterocyclic groups (with 3 fused rings) derived from carbazole (N₁), dibenzofuran (O₁), dibenzothiophene (S₁), carboline (N₂), perimidine (N₂), pyridoindole (N₂); and,
C₁₄heterocyclic groups (with 3 fused rings) derived from acridine (N₁), xanthene (O₁), thioxanthene (S₁), oxanthrene (O₂), phenoxathiin (O₁S₁), phenazine (N₂), phenoxazine (N₁O₁), phenothiazine (N₁S₁), thianthrene (S₂), phenanthridine (N₁), phenanthroline (N₂), phenazine (N₂).

Heteroaryl groups that have a nitrogen ring atom in the form of an —NH— group may be N-substituted, that is, as —NR—. For example, pyrrole may be N-methyl substituted, to give N-methylpyrrole. Examples of N-substitutents include C₁-C₇ alkyl; C₆-C₂₀ carboaryl; C₆-C₂₀ carboaryl-C₁-C₇ alkyl; C₁-C₇ alkyl-acyl; C₆-C₂₀ carboaryl-acyl; C₆-C₂₀ carboaryl-C₁-C₇ alkyl-acyl; etc.

Heteroaryl groups) which have a nitrogen ring atom in the form of an —N=group may be substituted in the form of an N-oxide, that is, as —N(→O)═ (also denoted —N⁺(→O⁻)═). For example, quinoline may be substituted to give quinoline N-oxide; pyridine to give pyridine N-oxide; benzofurazan to give benzofurazan N-oxide (also known as benzofuroxan).

### Molecular Weight

In one embodiment, the compound has a molecular weight of 250 to 1000.

In one embodiment, the bottom of range is 275; 300; 325; 350; 375; 400; 425; 450.

In one embodiment, the top of range is 900; 800; 700; 600; 500; 400.

In one embodiment, the range is 250 to 900.

In one embodiment, the range is 250 to 800.

In one embodiment, the range is 250 to 700.

In one embodiment, the range is 250 to 600.

In one embodiment, the range is 250 to 500.

### SOME PREFERRED EXAMPLES

All plausible and compatible combinations of the embodiments described above are explicitly disclosed herein. Each of these combinations is disclosed herein to the same extent as if each individual combination was specifically and individually recited.

Examples of some preferred compounds include the following:

| | |
|---|---|
| 1 | 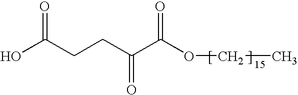 |
| 2 | 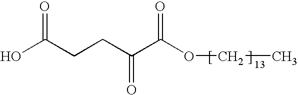 |
| 3 | 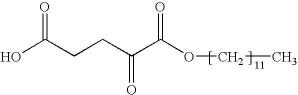 |
| 4 | 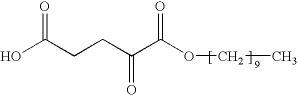 |
| 5 | 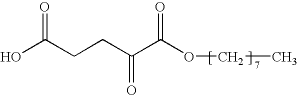 |
| 6 | 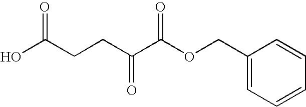 |
| 7 | 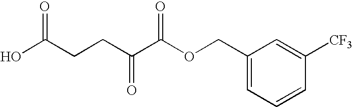 |
| 8 | 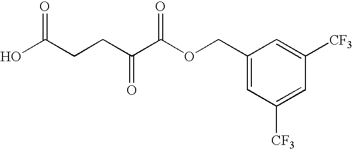 |
| 9 | 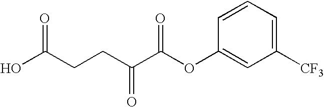 |
| 10 | 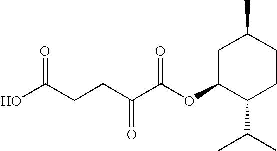 |
| 11 | 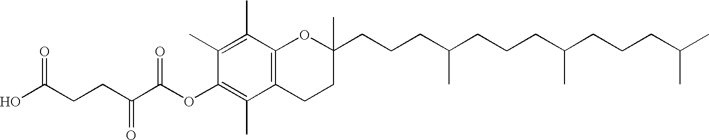 |

Additional Compounds—Compounds that Activate HIFα Hydroxylase

One aspect of the present invention pertains to compounds (generally) that activate HIFα hydroxylase (for example, HIFα prolyl hydroxylase), and their use in medicine.

The phrase “a compound that activates HIFα hydroxylase” pertains to a compound that increases the rate or level of HIFα hydroxylase activity whereby the HIFα hydroxylase activity is assessed by the amount of end-product HIF-1α, that is, an increase in the hydroxylation of HIF-1α. Thus, a decrease in HIF-1α protein levels may indicate activation of HIFα hydroxylase.

Suitable methods for determining HIFα hydroxylase activation are described herein and/or are well known it the art.

The increase in HIFα hydroxylase activity may be a low level increase of about 2 fold to 10 fold; a medium level increase of about 10 fold to 100 fold; or a high level increase of above about 100 fold.

HIFα hydroxylases have been described previously and are well known in the art. A preferred HIFα hydroxylase is HIFα prolyl hydroxylase. In mammalian cells, three isoforms have been identified, specifically, the prolyl hydroxylase domain (PHD) enzymes (PHD1, PHD2, PHD3), and were shown to hydroxylate HIFα in vitro. These enzymes have an absolute requirement for dioxygen as co-substrate. The overall reaction results in insertion of one oxygen atom into the HIFα peptide substrate at the prolyl residue, the other generating succinate from α-ketoglutarate with the release of CO₂.

In one embodiment, the compound acts (or additionally acts) as a substrate or co-factor for a HIFα hydroxylase, preferably HIFα prolyl hydroxylase.

In one embodiment, the compound that activates HIFα hydroxylase is (or additionally is) an α-ketoglutarate compound as described herein.

In one embodiment, the α-ketoglutarate compound described herein is (or additionally is) a compound that activates HIFα hydroxylase.

Additional Compounds—Compounds that Increase the Level of α-Ketoglutarate

One aspect of the present invention pertains to compounds (generally) that increase the level of α-ketoglutarate (e.g., in a cell), and their use in medicine.

For example, a compound may increase α-ketoglutarate levels by inhibiting other enzymes such as α-ketoglutarate dehydrogenase and/or branched-chain keto acid dehydrogenase. Blocking these enzymes will have a dual effect of increasing α-ketoglutarate levels and decreasing succinate levels.

Moreover, both enzymes are structural homologs that use lipoic acid as a cofactor. Therefore, a lipoic acid analogue may be another potential inhibitor of these enzymes, and so be a compound that increases the level of α-ketoglutarate

Alternatively, a compound might increase the level of α-ketoglutarate by enhancing glutamate oxaloacetate transaminase (GOT) activity. Glutamate itself will activate GOT activity leading to increased α-ketoglutarate levels.

Moreover, the compound may be selected from upstream metabolites of the TCA cycle including oxaloacetate, citrate, isocitrate, and derivatives thereof.

### Additional Compounds—α-Ketoglutarates Generally

One aspect of the present invention pertains to α-ketoglutaric acid, α-ketoglutarate salts, and α-ketoglutaric acid derivatives (e.g., esters of α-ketoglutaric acid, generally), and, especially, their use in medicine, for example, in the treatment of the conditions described herein.

In one embodiment, the compound is an α-ketoglutarate bearing (e.g., conjugated to, coupled to) an amino acid moiety (e.g., an α-amino acid moiety) (e.g., an ornithine or arginine moiety).

In one embodiment, the compound is an α-ketoglutarate ester (i.e., an ester of α-ketoglutaric acid) having an amino acid moiety (e.g., an α-amino acid moiety) (e.g., an ornithine or arginine moiety) that is, or is part of, an ester group (i.e., —C(═O)OR) formed from one of the acid groups of α-ketoglutaric acid.

Such compounds are known in the literature (see, e.g. Le Boucher et al. (1997)) and/or are commercially available and/or may be prepared using conventional synthetic procedures known to the skilled person.

### Isomers

Certain compounds may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r-forms; endo- and exo-forms; R—, S—, and meso-forms; D- and L-forms; d- and I-forms; (+) and (−) forms; keto-, enol-, and enolate-forms; syn- and anti-forms; synclinal- and anticlinal-forms; α- and α-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically, excluded from the term “isomers,” as used herein, are structural (or constitutional) isomers (i.e., isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amidelimino alcohol, amidine/amidine, nitroso/oxime, thioketone/enethiol, N-nitroso/hydroxyazo, and nitro/aci-nitro.

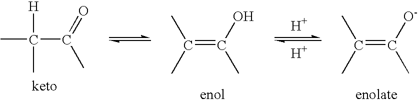

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof. Methods for the preparation (e.g., asymmetric synthesis) and separation (e.g., fractional crystallisation and chromatographic means) of such isomeric forms are either known in the art or are readily obtained by adapting the methods taught herein, or known methods, in a known manner.

### Salts

It may be convenient or desirable to prepare, purify, and/or handle a corresponding salt of the active compound, for example, a pharmaceutically-acceptable salt. Examples of pharmaceutically acceptable salts are discussed in Berge et al., 1977, “Pharmaceutically Acceptable Salts,” J. Pharm. Sci., Vol. 66, pp. 1-19.

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al⁺³. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group that may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

Unless otherwise specified, a reference to a particular compound also includes salt forms thereof.

### Solvates

It may be convenient or desirable to prepare, purify, and/or handle a corresponding solvate of the active compound. The term “solvate” is used herein in the conventional sense to refer to a complex of solute (e.g., active compound, salt of active compound) and solvent. If the solvent is water, the solvate may be conveniently referred to as a hydrate, for example, a mono-hydrate, a di-hydrate, a tri-hydrate, etc.

Unless otherwise specified, a reference to a particular compound also includes solvate forms thereof.

### Chemically Protected Forms

It may be convenient or desirable to prepare, purify, and/or handle the active compound in a chemically protected form. The term “chemically protected form” is used herein in the conventional chemical sense and pertains to a compound in which one or more reactive functional groups are protected from undesirable chemical reactions under specified conditions (e.g., pH, temperature, radiation, solvent, and the like). In practice, well known chemical methods are employed to reversibly render unreactive a functional group, which otherwise would be reactive, under specified conditions. In a chemically protected form, one or more reactive functional groups are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts; 3rd Edition; John Wiley and Sons, 1999).

Unless otherwise specified, a reference to a particular compound also includes chemically protected forms thereof.

A wide variety of such “protecting,” “blocking,” or “masking” methods are widely used and well known in organic synthesis. For example, a compound which has two nonequivalent reactive functional groups, both of which would be reactive under specified conditions, may be derivatized to render one of the functional groups “protected,” and therefore unreactive, under the specified conditions; so protected, the compound may be used as a reactant which has effectively only one reactive functional group. After the desired reaction (involving the other functional group) is complete, the protected group may be “deprotected” to return it to its original functionality.

For example, a hydroxy group may be protected as an ether (—OR) or an ester (—OC(═O)R), for example, as: a t-butyl ether; a benzyl, benzhydryl (diphenylmethyl), or trityl (triphenylmethyl)ether; a trimethylsilyl or t-butyldimethylsilyl ether; or an acetyl ester (—OC(═O)CH₃, —OAc).

For example, an aldehyde or ketone group may be protected as an acetal (R—CH(OR)₂) or ketal (R₂C(OR)₂), respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide (—NRCO—R) or a urethane (—NRCO—OR), for example, as: a methyl amide (—NHCO—CH₃); a benzyloxy amide (—NHCO—OCH₂C₆Hs, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulphonyl)ethyloxy amide (—NH-Psec); or, in suitable cases (e.g., cyclic amines), as a nitroxide radical (>N—O.).

For example, a carboxylic acid group may be protected as an ester for example, as: an C₁₋₇alkyl ester (e.g., a methyl ester; a t-butyl ester); a C₁₋₇haloalkyl ester (e.g., a C₁₋₇trihaloalkyl ester); a triC₁₋₇alkylsilyl-C₁₋₇alkyl ester; or a C₁₋₂₀aryl-C₁₋₇alkyl ester (e.g., a benzyl ester; a nitrobenzyl ester); or as an amide, for example, as a methyl amide.

For example, a thiol group may be protected as a thioether (—SR), for example, as: a benzyl thioether; an acetamidomethyl ether (—S—CH₂NHC(═O)CH₃).

### Prodrugs

It may be convenient or desirable to prepare, purify, and/or handle the active compound in the form of a prodrug. The term “prodrug,” as used herein, pertains to a compound which, when metabolised (e.g., in vivo), yields the desired active compound. Typically, the prodrug is inactive, or less active than the active compound, but may provide advantageous handling, administration, or metabolic properties.

Unless otherwise specified, a reference to a particular compound also includes prodrugs thereof.

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Also, some prodrugs are activated enzymatically to yield the active compound, or a compound which, upon further chemical reaction, yields the active compound (for example, as in ADEPT, GDEPT, LIDEPT, etc.). For example, the prodrug may be a sugar derivative or other glycoside conjugate, or may be an amino acid ester derivative.

### Chemical Synthesis

Several methods for the chemical synthesis of α-ketoglutarate esters are described herein. These and/or other well known methods may be modified and/or adapted in known ways in order to facilitate the synthesis of additional α-ketoglutarate esters and other compounds described herein, in accordance with standard techniques, from readily available starting materials, and using appropriate reagents and reaction conditions. If necessary and appropriate, the target compounds may be isolated from their reaction mixtures using conventional techniques, for example chromatography such as HPLC or FLASH chromatography.

For example, one general procedure for preparing α-ketoglutarate esters involves the alkylation (e.g., benzylation) of an α-keto acid or its derivative (see, e.g., Takeuchi et al., 1999; Natsugari et al., 1987). Another general procedure involves the esterification of an α-keto acid or its derivative (see, e.g., Hartenstein et al., 1993; Beyerman et al., 1961). Another general procedure involves an ester exchange (see, e.g., Domagala et al., 1980). The synthesis method may be single-step or multi-step.

The synthesis method may employ protective groups, for example, O-protecting groups, such as groups known to be suitable for protecting primary and/or secondary hydroxyl groups, for example, the O-protecting groups mentioned in “Protective Groups in Organic Chemistry”, edited by J. W. F. McOmie, Plenum Press (1973), and “Protective Groups in Organic Synthesis”, 3rd edition, T. W. Greene & P. G. M. Wutz, Wiley-Interscience (1999). Some preferred O-protecting groups include alkylcarbonyl and arylcarbonyl groups (e.g., acyl, e.g., benzoyl), triarylmethyl groups (e.g., triphenylmethyl (trityl) and dimethoxytrityl) and silyl groups (e.g., trialkylsilyl, such as trimethylsilyl).

### Compositions

One aspect of the present invention is a composition (e.g., pharmaceutical composition) comprising one or more compounds (e.g., α-ketoglutarate compounds; compounds that activate HIFα hydroxylase; compounds that activate PHD; compounds that inhibit or prevent HIF stabilization; compounds that increases the level of α-ketoglutarate, etc.) as described herein, and a pharmaceutically acceptable carrier.

### Uses

The compounds described herein (e.g., α-ketoglutarate compounds; compounds that activate HIFα hydroxylase; compounds that activate PHD; compounds that inhibit or prevent HIF stabilization; compounds that increases the level of α-ketoglutarate, etc.) are useful, for example, to activate PHD, to inhibit or prevent HIF stabilization, in the treatment of hypoxia-induced angiogenesis, and in the treatment of diseases and conditions that are mediated by hypoxia-induced angiogenesis.

### Use in Methods of Activating PHD

One aspect of the present invention pertains to a method of activating PHD in a cell, in vitro or in vivo, comprising contacting the cell with an effective amount of a compound (e.g., a α-ketoglutarate compound; a compound that activates HIFα hydroxylase; a compound that activates PHD; a compound that inhibits or prevents HIF stabilization; a compound that increases the level of α-ketoglutarate, etc.), as described herein.

Suitable methods for determining PHD activation are described herein and/or are well known in the art.

### Use in Methods of Inhibiting HIF Stabilization

One aspect of the present invention pertains to a method of inhibiting or preventing HIF stabilization in a cell, in vitro or in vivo, comprising contacting the cell with an effective amount of a compound (e.g., a α-ketoglutarate compound; a compound that activates HIFα hydroxylase; a compound that activates PHD; a compound that inhibits or prevents HIF stabilization; a compound that increases the level of α-ketoglutarate, etc.), as described herein.

Suitable methods for determining HIF stabilization are described herein and/or are well known in the art.

### Use in Methods of Activating HIFα Hydroxylase

One aspect of the present invention pertains to a method of activating HIFα hydroxylase in a cell, in vitro or in vivo, comprising contacting the cell with an effective amount of a compound (e.g., a α-ketoglutarate compound; a compound that activates HIFα hydroxylase; a compound that activates PHD; a compound that inhibits or prevents HIF stabilization; a compound that increases the level of α-ketoglutarate, etc.), as described herein.

Suitable methods for determining HIFα hydroxylase activation are described herein and/or are well known in the art.

### Use in Methods of Inhibiting Cell Proliferation, Etc.

One aspect of the present invention pertains to a method of (a) regulating (e.g., inhibiting) cell proliferation (e.g., proliferation of a cell), (b) inhibiting cell cycle progression, (c) promoting apoptosis, or (d) a combination of one or more these, in vitro or in vivo, comprising contacting cells (or the cell) with an effective amount of a compound (e.g., an α-ketoglutarate compound; a compound that activates HIFα hydroxylase; a compound that increases the level of α-ketoglutarate), as described herein.

In one embodiment, the method is a method of regulating (e.g., inhibiting) cell proliferation (e.g., proliferation of a cell), in vitro or in vivo, comprising contacting cells (or the cell) with an effective amount of a compound (e.g., a α-ketoglutarate compound; a compound that activates HIFα hydroxylase; a compound that activates PHD; a compound that inhibits or prevents HIF stabilization; a compound that increases the level of α-ketoglutarate, etc.), as described herein.

In one embodiment, the method is performed in vitro.

In one embodiment, the method is performed in vivo.

In one embodiment, a compound is provided in the form of a pharmaceutically acceptable composition.

Any type of cell may be treated, including but not limited to, lung, gastrointestinal (including, e.g., bowel, colon), breast (mammary), ovarian, prostate, liver (hepatic), kidney (renal), bladder, pancreas, brain, and skin.

One of ordinary skill in the art is readily able to determine whether or not a candidate compound regulates (e.g., inhibits) cell proliferation, etc. For example, assays that may conveniently be used to assess the activity offered by a particular compound are described herein.

For example, a sample of cells (e.g., from a tumour) may be grown in vitro and a α-ketoglutarate compound brought into contact with said cells, and the effect of the compound on those cells observed. As an example of “effect,” the morphological status of the cells (e.g., alive or dead, etc.) may be determined. Where the compound is found to exert an influence on the cells, this may be used as a prognostic or diagnostic marker of the efficacy of the compound in methods of treating a patient carrying cells of the same cellular type.

### Use in Methods of Therapy

Another aspect of the present invention pertains to a compound (e.g., a α-ketoglutarate compound; a compound that activates HIFα hydroxylase; a compound that activates PHD; a compound that inhibits or prevents HIF stabilization; a compound that increases the level of α-ketoglutarate, etc.), as described herein, for use in a method of treatment of the human or animal body by therapy.

### Use in the Manufacture of Medicaments

Another aspect of the present invention pertains to use of a compound (e.g., a α-ketoglutarate compound; a compound that activates HIFα hydroxylase; a compound that activates PHD; a compound that inhibits or prevents HIF stabilization; a compound that increases the level of α-ketoglutarate, etc.), as described herein, in the manufacture of a medicament for use in treatment (e.g., of a condition as described herein).

### Methods of Treatment

Another aspect of the present invention pertains to a method of treatment comprising administering to a patient in need of treatment a therapeutically effective amount of a compound (e.g., a α-ketoglutarate compound; a compound that activates HIFα hydroxylase; a compound that activates PHD; a compound that inhibits or prevents HIF stabilization; a compound that increases the level of α-ketoglutarate, etc.) as described herein, preferably in the form of a pharmaceutical composition.

### Conditions Treated—Conditions Encountering Hypoxic Conditions

In one embodiment, the treatment is treatment of a condition that encounters hypoxic conditions as it proceeds (e.g., as solid tumours grow).

In one embodiment, the treatment is treatment of a condition selected from: cancer, psoriasis, atherosclerosis, menorrhagia, endometrosis, arthritis (both inflammatory and rheumatoid), macular degeneration, Paget's disease, retinopathy and its vascular complications (including proliferative and diabetic retinopathy), benign vascular proliferation, fibroses, obesity and inflammation.

It is very well known in the art that such conditions encounter hypoxic conditions as they proceed. This is especially true for those cancers characterised by solid tumours.

### Conditions Treated—Angiogenesis

The compounds described herein are useful in the treatment of (e.g., inhibition on angiogenesis (as “anti-angiogenesis agents”).

In one embodiment, the treatment is treatment of angiogenesis (e.g., inhibition of angiogenesis), or treatment of a condition that is characterised by inappropriate, excessive, and/or undesirable angiogenesis.

In one embodiment, the angiogenesis is hypoxia-induced angiogenesis.

In one embodiment, the angiogenesis is angiogenesis in which the activity of HIF-1α is upregulated due to hypoxia.

In one embodiment, the treatment is treatment of a condition characterised by hypoxia-induced angiogenesis.

In one embodiment, the treatment is treatment of: cancer, psoriasis, atherosclerosis, menorrhagia, endometrosis, arthritis (both inflammatory and rheumatoid), macular degeneration, Paget's disease, retinopathy and its vascular complication's (including proliferative and diabetic retinopathy), benign vascular proliferation, fibroses, obesity, or inflammation.

### Conditions Treated—Proliferative Conditions and Cancer

The compounds described herein are useful in the treatment of proliferative conditions (as “anti-proliferative agents”), cancer (as “anti-cancer agents”), etc.

As used herein, the term “antiproliferative agent” pertains to a compound that treats a proliferative condition (i.e., a compound which is useful in the treatment of a proliferative condition). The terms “proliferative condition,” “proliferative disorder,” and “proliferative disease,” are used interchangeably herein and pertain to an unwanted or uncontrolled cellular proliferation of excessive or abnormal cells that is undesired, such as, neoplastic or hyperplastic growth.

As used herein, the term “anticancer agent” pertains to a compound that treats a cancer (i.e., a compound which is useful in the treatment of a cancer). The anti-cancer effect may arise through one or more mechanisms, including but not limited to, the regulation of cell proliferation, the inhibition of cell cycle progression, the inhibition of angiogenesis (the formation of new blood vessels), the inhibition of metastasis (the spread of a tumour from its origin), the inhibition of invasion (the spread of tumour cells into neighbouring normal structures), or the promotion of apoptosis (programmed cell death).

One of ordinary skill in the art is readily able to determine whether or not a candidate compound treats a proliferative condition, or treats cancer, for any particular cell type. For example, assays that may conveniently be used to assess the activity offered by a particular compound are described herein.

Note that active compounds includes both compounds with intrinsic activity (drugs) as well as prodrugs of such compounds, which prodrugs may themselves exhibit little or no intrinsic activity.

In one embodiment, the treatment is treatment of a proliferative condition.

In one embodiment, the treatment is treatment of a proliferative condition characterised by benign, pre-malignant, or malignant cellular proliferation, including but not limited to, neoplasms, hyperplasias, and tumours (e.g., histocytoma, glioma, astrocyoma, osteoma), cancers (see below), psoriasis, bone diseases, fibroproliferative disorders (e.g., of connective tissues), pulmonary fibrosis, atherosclerosis, smooth muscle cell proliferation in the blood vessels, such as stenosis or restenosis following angioplasty.

In one embodiment, the treatment is treatment of cancer.

In one embodiment, the treatment is treatment of: lung cancer, small cell lung cancer, non-small cell lung cancer, gastrointestinal cancer, stomach cancer, bowel cancer, colon cancer, rectal cancer, colorectal cancer, thyroid cancer, breast cancer, ovarian cancer, endometrial cancer, prostate cancer, testicular cancer, liver cancer, kidney cancer, renal cell carcinoma, bladder cancer, pancreatic cancer, brain cancer, glioma, sarcoma, osteosarcoma, bone cancer, skin cancer, squamous cancer, Kaposi's sarcoma, melanoma, malignant melanoma, lymphoma, or leukemia.

In one embodiment, the treatment is treatment of:
a carcinoma, for example a carcinoma of the bladder, breast, colon (e.g., colorectal carcinomas such as colon adenocarcinoma and colon adenoma), kidney, epidermal, liver, lung (e.g., adenocarcinoma, small cell lung cancer and non-small cell lung carcinomas), oesophagus, gall bladder, ovary, pancreas (e.g., exocrine pancreatic carcinoma), stomach, cervix, thyroid, prostate, skin (e.g., squamous cell carcinoma);
a hematopoietic tumour of lymphoid lineage, for example leukemia, acute lymphocytic leukemia, B-cell lymphoma, T-cell lymphoma, Hodgkin's lymphoma, non-Hodgkin's lymphoma, hairy cell lymphoma, or Burkett's lymphoma;
a hematopoietic tumour of myeloid lineage, for example acute and chronic myelogenous leukemias, myelodysplastic syndrome, or promyelocytic leukemia;
a tumour of mesenchymal origin, for example fibrosarcoma or habdomyosarcoma;
a tumour of the central or peripheral nervous system, for example astrocytoma, neuroblastoma, glioma or schwannoma;
melanoma; seminoma; teratocarcinoma; osteosarcoma; xenoderoma pigmentoum; keratoctanthoma; thyroid follicular cancer; or Kaposi's sarcoma.

In one embodiment, the treatment is treatment of solid tumour cancer (e.g., cancer characterized by the appearance of solid tumours).

In one embodiment, the treatment is treatment of cancer selected from: phaeochromocytoma, paraganglioma, leiomyoma, renal cell carcinoma, gastric carcinoma, and colorectal carcinoma.

In one embodiment, the treatment is treatment of cancer (e.g., tumours) characterised by (e.g., that exhibits) SDH dysfunction.

In one embodiment, the treatment is treatment of cancer that develops SDH down-regulation in a later stage of the disease.

In one embodiment, the treatment is treatment of gastric or colorectal cancer, for example, Dukes' stage C of colorectal cancer.

In one embodiment, the treatment is treatment of oral carcinoma tumours. (Lower expression of SDH genes has been observed in oral tumours upon the transition from adenoma to carcinoma.)

The compounds described herein may be used in the treatment of the cancers described herein, independent of the mechanisms discussed herein.

In one embodiment, the treatment is treatment as described herein, wherein the patient has inherited or somatic mutations in subunits A, B, C or D of the SDH gene or FH or down regulation of the expression of any of the SDH genes (subunits A, B, C or D) or of FH or impaired activity of the enzymes encoded by said genes.

Conditions Treated—Cancer with HIF-1α Up-Regulation

In one embodiment, the treatment is treatment of cancer (e.g., as described herein) in which the activity of HIF-1α is upregulated due to hypoxia.

Conditions Treated—Cancer with TCA Cycle Enzyme Down-Regulation

In one embodiment, the treatment is treatment of cancer (e.g., as described herein) in which the activity of one of the enzymes of the TCA cycle is down-regulated.

Without wishing to be bound by any particular theory, it is believed that the down-regulation of one of the enzymes of the TCA cycle results inevitably in an increase in the level of succinate.

Examples of enzymes of the TCA cycle include, for example, succinate dehydrogenase (SDH) and fumarate hydratase (FH).

The phrase “down-regulation of SDH” is intended to include, for example, a decrease of SDH activity due to mutations in one or more of the SDH genes; or due to a reduction in the expression of one of these genes (for example by promoter methylation); or due to other indirect effects such as mutations in mitochondrial DNA, or an increase in the levels of an endogenous compound that negatively regulates SDH function (for example fumarate (due to FH mutations), reactive oxygen species, and a protein that binds to and inhibits SDH activity).

### Treatment

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, alleviatiation of symptoms of the condition, amelioration of the condition, and cure of the condition. Treatment as a prophylactic measure (i.e., prophylaxis) is also included. For example, use with patients who have not yet developed the condition, but who are at risk of developing the condition, is encompassed by the term “treatment.”

For example, treatment of cancer includes the prophylaxis of cancer, reducing the incidence of cancer, alleviating the symptoms of cancer, etc.

The term “therapeutically-effective amount,” as used herein, pertains to that amount of an active compound, or a material, composition or dosage form comprising an active compound, which is effective for producing some desired therapeutic effect, commensurate with a reasonable benefit/risk ratio, when administered in accordance with a desired treatment regimen.

### Combination Therapies—Generally

The term “treatment” includes combination treatments and therapies, in which two or more treatments or therapies are combined, for example, sequentially or simultaneously. For example, the compounds described herein may also be used in combination therapies, e.g., in conjunction with other agents, for example, cytotoxic agents, anticancer agents, etc. Examples of treatments and therapies include, but are not limited to, chemotherapy (the administration of active agents, including, e.g., drugs, antibodies (e.g., as in immunotherapy), prodrugs (e.g., as in photodynamic therapy, GDEPT, ADEPT, etc.); surgery; radiation therapy; photodynamic therapy; gene therapy; and controlled diets.

The particular combination would be at the discretion of the physician who would select dosages using his common general knowledge and dosing regimens known to a skilled practitioner.

The agents (e.g., a α-ketoglutarate compound; a compound that activates HIFα hydroxylase; a compound that activates PHD; a compound that inhibits or prevents HIF stabilization; a compound that increases the level of α-ketoglutarate, etc.; plus one or more other agents) may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g., 1, 2, 3, 4 or more hours apart, or even longer periods apart where required), the precise dosage regimen being commensurate with the properties of the therapeutic agent(s).

The agents may be formulated together in a single dosage form, or alternatively, the individual agents may be formulated separately and presented together in the form of a kit, optionally with instructions for their use, as described below.

### Combination Therapies—Enhancer of ALA Synthase

Without wishing to be bound to any particular theory, it is believed that metabolism of α-ketoglutarate to succinate in the TCA cycle results in the formation of the intermediate succinyl-CoA. Delta-ALA is produced from succinyl CoA and glycine by the enzyme ALA synthase. ALA is converted to the protoporphyrin IX (PpIX) followed by the synthesis of haem by the enzymes of the haem biosynthetic pathway. High concentrations of porphyrin ring compounds are known to be toxic as they can release singlet oxygen, for example when exposed to light.

Again, without wishing to be bound to any particular theory, it is believed that activation of ALA synthase diverts metabolism of α-ketoglutarate via succinyl-CoA towards the haem pathway and can induce cell death.

Thus, in one embodiment (e.g., of use in methods of therapy, of use in the manufacture of medicaments, of methods of treatment), two agents are employed: (a) a first agent (e.g., an α-ketoglutarate compound; a compound that activates HIFα hydroxylase; a compound that increases the level of α-ketoglutarate), and (b) a second agent.

For example, in one embodiment, the method of treatment is a method of treatment comprising co-administering to a patient in need of such treatment an effective amount of (a) a first agent (e.g., an α-ketoglutarate compound; a compound that activates HIFα hydroxylase; a compound that increases the level of α-ketoglutarate), and (b) a second agent.

For example, in one embodiment, the use is use of (a) a first agent (e.g., an α-ketoglutarate compound; a compound that activates HIFα hydroxylase; a compound that increases the level of α-ketoglutarate), and (b) a second agent, in the manufacture of medicament for treatment.

The first and second agents may be administered separately, sequentially, or simultaneously.

In one embodiment, the second agent is a compound that is an enhancer of aminolaevulinic acid (ALA) synthase.

As used herein, the term “enhancer” pertains to a compound that increases the rate or level of the activity of ALA synthase. The increase can be a low level increase of about 2 fold to 10 fold; a medium level increase of about 10 fold to 100 fold; or a high level increase of above 100 fold.

In one embodiment, the second agent is selected from: barbiturates, anticonvulsants, non-narcotic analgetics, and non-steriodal anti-inflammatory compounds.

In one embodiment, the second agent is selected from: Allyl isopropyl acetamide, Phenobarbital, Deferoxamine, Felbamate, Lamotrigine, Tiagabine, Cyclophosphamide, N-methylprotoporphyrin, Succinyl-acetone, Carbamazepine, Ethanol, Phenyloin, Azapropazone, Chloroquine, Paracetamol, Griseofulvin, Cadmium, Iron, Pyridoxine. (These compounds are well known in the art to induce or activate ALA synthase.)

In one embodiment, the second agent is selected from: Ethosuximide, Diazepam, Hydantoins, Methsuximide, Paramethadione, Phenobarbitone, Phensuximide, Phenyloin, Primidone, Succinimides, Bromides, Aspirin, Dihydroergotamine-Mesylate, Ergotamine Tartrate, Chloramphenicol, Dapsone, Erythromycin, Flucloxacillin, Pyrazinamide, Sulphonamides, Ampicillin, Vancomycin, Sulphonylureas Glipizidelnsulin, Alpha tocopheryl acetate, Ascorbic Acid, Folic Acid, Fructose, Glucose, Haem Arginate, Amidopyrine, Dichloralphenazone, Diclofenac Na, Dipyrone, Oxyphenbutazone, Propyphenazone, Aspitin, Codeine PO4, Dihydrocodeine, Canthaxanthin, β Carotene.

### Combination Therapies—Photodynamic Therapy

The methods of therapy described herein may further comprise the step of subjecting the patient to photodynamic therapy.

Photodynamic therapy (PDT) is a treatment that relies on the interaction between light and a substance in order to make cells more sensitive to light (photosensitiser). Following the absorption of light (photons), the photosensitiser transfers energy from the light to molecular oxygen, thereby generating reactive oxygen species (ROS). The biological responses to the photosensitiser are activated only in the particular areas of tissue that have been exposed to light.

PDT mediates cell death by a variety of mechanisms, including: (i) the ROS that are generated by PDT can kill tumour cells directly; (ii) PDT damages the tumour-associated vasculature, and (iii) PDT can activate an immune response against tumour cells (Dolmans et al., 2003).

PDT may use a photosensitiser that has been administered to the patient, for example, the photosensitiser Photofrin used for the prophylactic treatment of bladder cancer, or 5-aminolevulinic acid (5-ALA or Levulan (DUSA Pharma)). Molecules that are endogenous to a cell or tissue (such as the above mentioned ALA) may also be employed as photosensitiesers.

A compound that increases α-ketoglutarate levels will selectively increase the levels of succinyl-CoA in cells where SDH is down regulated, because in these cells succinyl CoA cannot be further processed by the TCA. Therefore, treating these cells with a compound that enhances ALA synthase activity (and a compound that increases α-ketoglutarate levels) leads to increased levels of ALA and PpIX and thus apoptosis in cells exposed to PDT.

Therefore, without being bound by any particular theory, it is believed that cells with mutated or reduced SDH activity that have been treated with a combination of α-ketoglutarate and an ALA synthase stimulating compound, will have increased levels of porphyrin compounds compared to normal cells, thereby sensitising SDH inhibited cells to light.

Thus, one aspect of the present invention is a method of treatment, as described herein, further comprising the step of subjecting the patient to photodynamic therapy.

Another aspect of the present invention is a method of (e.g., PDT) treatment comprising the steps of: (i) simultaneous, separate, or sequential administration of (a) a first agent (e.g., a α-ketoglutarate compound; a compound that activates HIFα hydroxylase; a compound that activates PHD; a compound that inhibits or prevents HIF stabilization; a compound that increases the level of α-ketoglutarate, etc.), and (b) a photosensitizer (e.g., Photofrin; 5-ALA; an ALA synthase stimulating compound; etc.); followed by (ii) light irradiation.

Thus, one aspect of the present invention is a method of PDT treatment comprising the steps of: (i) simultaneous, separate, or sequential administration of (a) an α-ketoglutarate compound or a compound that activates HIFα hydroxylase or a compound that increases the level of α-ketoglutarate, and (b) a photosensitizer (e.g., Photofrin; 5-ALA; an ALA synthase stimulating compound; etc.); followed (ii) by light irradiation.

The light irradiation may be applied to the body as a whole or locally to a patient using any apparatus which can, respectively, irradiate the whole body of the patient or can irradiate locally with an appropriate wavelength and dose.

As used herein, the term “locally” means that only a part or parts of the body of a patient is irradiated. By local irradiation, it is possible to activate ROS in the part or parts of the body to be treated.

An advantage over known compounds and PDT methods is that by relying on SDH dysfunction in the cells that are to be treated, photosensitisation will occur in these cells only, due to the excess of succinyl CoA caused by SDH dysfunction. Therefore, while α-ketoglutarate and/or an ALA synthase inducer are be administered systemically, photosensytisation will be enhanced to significant levels only locally, in the tumours being treated/irradiated.

### Other Uses

The compounds described herein may also be used as cell culture additives to activatve HIFα hydroxylase, to activate PHD, to inhibit or prevent HIF stablilization, to increase the level of α-ketoglutarate, to inhibit cell proliferation, etc.

The compounds described herein may also be used as part of an in vitro assay, for example, in order to determine whether a candidate host is likely to benefit from treatment with the compound in question.

The compounds described herein may also be used as a standard, for example, in an assay, in order to identify other active compounds.

### Kits

One aspect of the invention pertains to a kit comprising (a) an active compound as described herein, or a composition comprising an active compound as described herein, e.g., preferably provided in a suitable container and/or with suitable packaging; and (b) instructions for use, e.g., written instructions on how to administer the active compound or composition.

The written instructions may also include a list of indications for which the active ingredient is a suitable treatment.

In one embodiment, the kit further comprises a second agent, for example, as described herein.

### Routes of Administration

The active compound or pharmaceutical composition comprising the active compound may be administered to a subject by any convenient route of administration, whether systemic ally/peripherally or topically (i.e., at the site of desired action).

Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### The Subject/Patient

The subject/patient may be a chordate, a vertebrate, a mammal, a placental mammal, a marsupial (e.g., kangaroo, wombat), a monotreme (e.g., duckbilled platypus), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutans, gibbon), or a human.

Furthermore, the subject/patient may be any of its forms of development, for example, a foetus.

In one preferred embodiment, the subject/patient is a human.

### Formulations and Administration

The compound may be formulated into a pharmaceutical composition, such as by mixing with one or more of a suitable carrier, diluent or excipient, by using techniques that are known in the art.

There may be different composition/formulation requirements dependent on the different delivery systems. By way of example, the compound may be formulated to be administered using a mini-pump or by a mucosal route, for example, as a nasal spray or aerosol for inhalation or ingestible solution, or parenterally in which the composition is formulated by an injectable form, for delivery, by, for example, an intravenous, intramuscular or subcutaneous route. Alternatively, the compound may be designed to be administered by a number of routes.

The routes for administration (delivery) include, but are not limited to, one or more of: oral (e.g., as a tablet, capsule, or as an ingestable solution), topical, mucosal (e.g., as a nasal spray or aerosol for inhalation), nasal, parenteral (e.g., by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, vaginal, epidural, sublingual.

Where the compound is to be administered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the compound can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring compounds, or they can be injected parenterally, for example intravenously, intramuscularly or subcutaneously. For parenteral administration, the compound may be best used in the form of a sterile aqueous solution that may contain other substances, for example enough salts or monosaccharides to make the solution isotonic with blood. For buccal or sublingual administration the compositions may be administered in the form of tablets or lozenges that can be formulated in a conventional manner.

If the pharmaceutical is a tablet, then the tablet may contain excipients such as microcrystalline cellulose, lactose, sodium citrate, calcium carbonate, dibasic calcium phosphate, and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating compounds such as magnesium stearate, stearic acid, glyceryl behenate, and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compound may be combined with various sweetening or flavouring compounds, colouring matter or dyes, with emulsifying and/or suspending compounds and with diluents such as water, ethanol, propylene glycol and glycerin, and combinations thereof.

If the compound is administered parenterally, then examples of such administration include one or more of: intravenously, intra-arterially, intraperitoneally, intrathecally, intraventricularly, intraurethrally, intrasternally, intracranially, intramuscularly or subcutaneously administering the component; and/or by using infusion techniques.

For parenteral administration, the compound is best used in the form of a sterile aqueous solution that may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

As indicated, the compound may be administered intranasally or by inhalation and is conveniently delivered in the form of a dry powder inhaler or an aerosol spray presentation from a pressurised container, pump, spray or nebuliser with the use of a suitable propellant, e.g., dichlorodifluoromethane, trichlorofluoromethane, dichlorotetrafluoroethane, a hydrofluoroalkane such as 1,1,1,2-tetrafluoroethane (HFA 134ATM) or 1,1,1,2,3,3,3-heptafluoropropane (HFA 227EATM), carbon dioxide, or other suitable gas. In the case of a pressurised aerosol, the dosage unit may be determined by providing a valve to deliver a metered amount. The pressurised container, pump, spray, or nebuliser may contain a solution or suspension of the compound, e.g., using a mixture of ethanol and the propellant as the solvent, which may additionally contain a lubricant, e.g., sorbitan trioleate. Capsules and cartridges (made, for example, from gelatin) for use in an inhaler or insufflator may be formulated to contain a powder mix of the compound and a suitable powder base such as lactose or starch.

Alternatively, the compound may be administered in the form of a suppository or pessary, or it may be applied topically in the form of a gel, hydrogel, lotion, solution, cream, ointment or dusting powder. The compound may also be dermally or transdermally administered, for example, by the use of a skin patch. The compound may also be administered by the pulmonary or rectal routes. The compound may also be administered by the ocular route. For ophthalmic use, the compound can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the compound may be formulated as a suitable ointment containing the compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, the compound can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol, and water.

The compound may also be administered via the peripheral blood, for example by using skin patches.

### Dosage

It will be appreciated by one of skill in the art that appropriate dosages of the active compounds, and compositions comprising the active compounds, can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action that achieves the desired effect without causing substantial harmful or deleterious side-effects.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, a suitable dose of the active compound is in the range of about 100 μg to about 250 mg (more typically about 100 μg to about 25 mg) per kilogram body weight of the subject per day. Where the active compound is a salt, an ester, an amide, a prodrug, or the like, the amount administered is calculated on the basis of the parent compound and so the actual weight to be used is increased proportionately.

### EXAMPLES

The following are examples are provided solely to illustrate the present invention and are not intended to limit the scope of the invention, as described herein.

### Chemical Synthesis

### Synthesis 1

### 2-(2-Carboxy-ethyl)-[1,3]dithiane-2-carboxylic acid ethyl

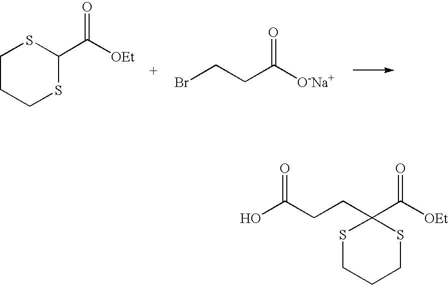

A solution of ethyl 1,3-dithiane carboxylate and one equivalent sodium 3-bromopropane carboxylate in DMF was added slowly to a well-stirred suspension of one equivalent of sodium hydride in dry toluene cooled to 5° C. The mixture was stirred in an ice bath for 1 hour and then stirred at room temperature for 12 hours. The toluene layer was extracted three times with 20 mL portions of water, dried over MgSO₄, filtered, and concentrated. The crude product is suitable for desulphurization or conversion to the α-keto ester; however, the product was purified by chromatography. See, for example, Eliel et al., 1978.

### Synthesis 2

### 2-Oxo-pentanedioic acid 1-ethyl ester

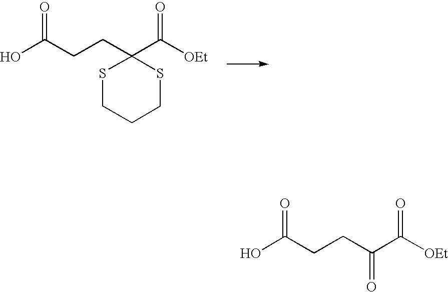

A solution of 2-(2-carboxy-ethyl)-[1,3]dithiane-2-carboxylic acid ethyl in acetonitrile was added quickly to a well-stirred solution of 4 equivalents of N-chlorosuccinimide and silver nitrate (4.5 equivalents) in aqueous 80% acetonitrile at 25° C. Silver chloride separated immediately as a voluminous white precipitate and the liquid phase became yellow. The mixture was stirred for 5-10 minutes and treated successively at 1 minute intervals with saturated aqueous sodium sulphite, saturated aqueous sodium carbonate, and brine; 1:1 hexane/dichloromethane was added; and the mixture was filtered. After the filter cake was washed thoroughly with 1:1 hexane/dichloromethane, the organic phase of the filtrate was dried (MgSO₄) and freed of solvent. See, for example, Corey et al., 1971.

### Synthesis 3

### 2-Oxo-pentanedioic acid

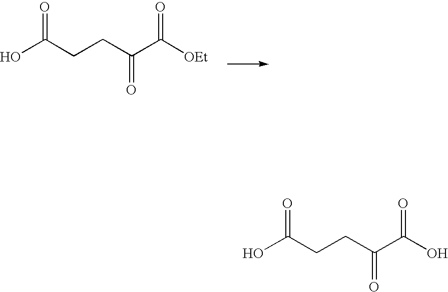

2-Oxo-pentanedioic acid 1-ethyl ester was dissolved in aqueous NaOH (3.5 equivalents) and ethanol and heated to reflux for 4 hours. The solvents were removed under reduced pressure and the residue was taken up in water and carefully acidified to pH 1 with conc. HCl. The acid was extracted with ether and the combined organics were dried (MgSO₄). See, for example, Schwetlick et al., 2001.

### Synthesis 4

### 2-Oxo-pentanedioic acid 1-(3-trifluoromethyl-phenyl)ester

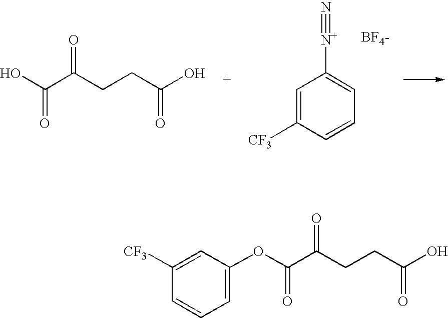

2-oxo-pentanedioic acid was dissolved in THF under nitrogen. To this solution was added one equivalent of NaH followed by one equivalent of chlorotrimethylsilane. The resulting trimethylsilyl ester of the α-keto acid was then treated dropwise with one equivalent of 3-trifluoromethyl-benzenediazonium tetrafluoroborate (for method of preparation, see, e.g., Starkey, 1943) and the reaction mixture was placed in an ultrasound bath to produce 2-oxo-pentanedioic acid 1-(3-trifluoromethyl-phenyl)ester. See, e.g., Olah, G. A., et al., 1991).

### Synthesis 5

### 2-Oxo-pentanedioic acid 1-((1S,2R,5S)-2-isopropyl-5-methyl-cylcohexyl)ester

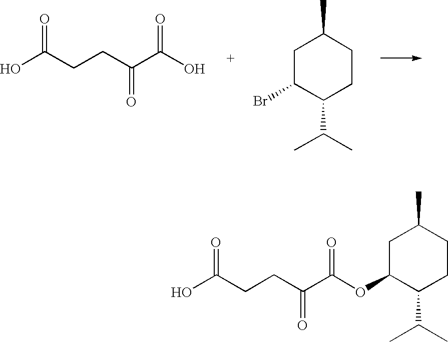

2-oxo-pentanedioic acid was dissolved in THF under nitrogen. To this solution was added one equivalent of NaH. The resulting sodium salt of the α-keto acid was then treated dropwise with one equivalent of (1R,2R,4S)-2-bromo-1-isopropyl-4-methyl-cyclohexane over a period of 100 hours. See, for example, Domagala, 1980.

### Synthesis 6

### 2-Oxo-pentanedioic acid 1-octyl ester

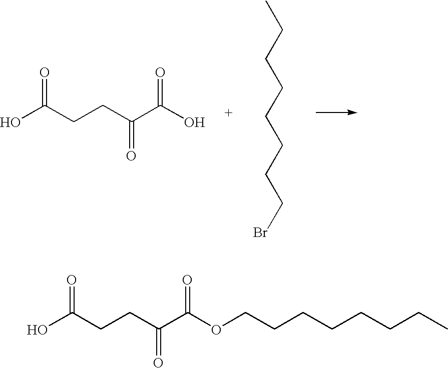

2-Oxo-pentanedioic acid was dissolved in THF under nitrogen. To this solution was added one equivalent of NaH. The resulting sodium salt of the α-keto acid was then treated dropwise with one equivalent of 1-octyl bromide over a period of 100 hours. See, for example, Domagala, 1980. Alternatively, octyl chloroformate was added drop-wise to a solution of α-ketoglutaric acid (10 mmol) and triethylamine (1.0 eq.) in dichloromethane (50 mL) at ambient temperature. The resulting mixture was stirred at ambient temperature for 16 hours and then diluted with dichloromethane (50 mL), washed with 0.5 N aqueous hydrochloric acid (50 mL), dried (magnesium sulphate), and the solvent removed under reduced pressure.

### Synthesis 7

### 2-Oxopentanedioic acid 1-(3-trifluoromethyl-benzyl)ester

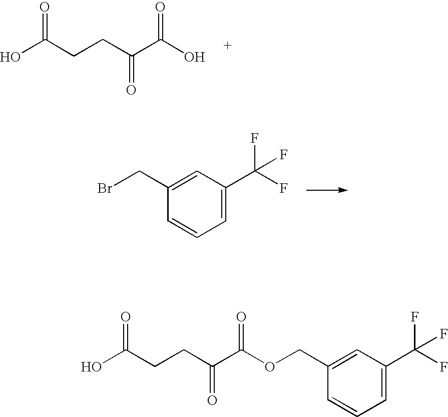

2-oxo-pentanedioic acid was dissolved in THF under nitrogen. To this solution was added one equivalent of NaH. The resulting sodium salt of the α-keto acid was then treated dropwise with one equivalent of benzyl-bromide to produce 2-oxopentanedioic acid 1-(3-trifluoromethyl-benzyl)ester. See, for example, Natsugari et al., 1987. Alternatively, benzyl bromide was added (1.1 eq.) to a solution of α-ketoglutaric acid (10 mmol) and dicyclohexylamine (1.2 eq.) in dimethyl formamide (50 mL) and the solution heated at 50° C. for 16 hours. The mixture was concentrated under reduced pressure and the residue re-suspended in dichloromethane (100 mL) and washed with 0.5 N aq. hydrochloric acid (50 mL). The organic layer was dried (magnesium sulphate) and the solvent removed under reduced pressure. The crude products were purified using normal phase flash column chromatography (hexane/ethyl acetate).

### Synthesis 8

### 2-Oxo-pentanedioic acid 1-benzyl ester

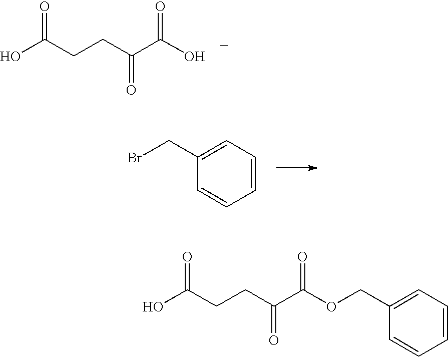

2-oxo-pentanedioic acid was dissolved in THF under nitrogen. To this solution was added one equivalent of NaH. The resulting sodium salt of the α-keto acid was then treated dropwise with one equivalent of benzyl-bromide to produce 2-oxo-pentanedioic acid 1-benzyl ester. See, for example, Natsugari et al., 1987. Alternatively, benzyl bromide was added (1.1 eq.) to a solution of α-ketoglutaric acid (10 mmol) and dicyclohexylamine (1.2 eq.) in dimethyl formamide (50 mL) and the solution heated at 50° C. for 16 hours. The mixture was concentrated under reduced pressure and the residue re-suspended in dichloromethane (100 mL) and washed with 0.5 N aqueous hydrochloric acid (50 mL). The organic layer was dried (magnesium sulphate) and the solvent removed under reduced pressure. The crude products were purified using normal phase flash column chromatography (hexane/ethyl acetate).

### Synthesis 9

### 2-oxo-pentanedioic acid 1-dodecyl ester

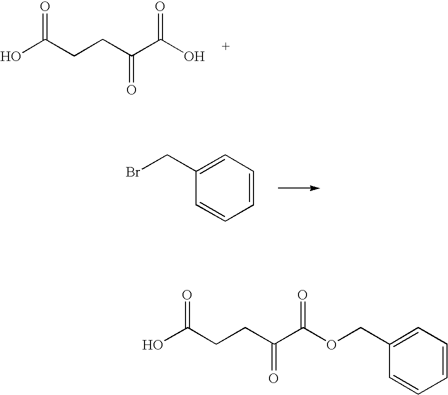

2-oxo-pentanedioic acid was dissolved in dichloromethane under nitrogen. To this solution was added one equivalent of NaH. The resulting sodium salt of the α-keto acid was then treated dropwise with one equivalent of 1-dodecyl-bromide over a period of 100 hours. See, e.g., Domogala, 1980.

### Synthesis 10

### 2-Oxo-pentanedioic acid 1-(3,5-bis-trifluoromethyl-benzyl)ester

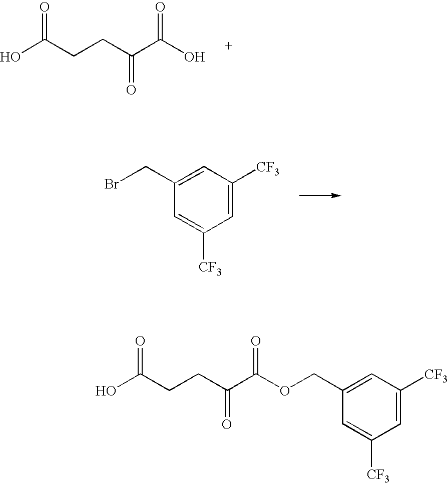

2-Oxo-pentanedioic acid was dissolved in THF under nitrogen. To this solution was added one equivalent of NaH. The resulting sodium salt of the α-keto acid was then treated dropwise with one equivalent of 3,5-di-(trifluoromethyl)benzyl bromide to produce 2-oxo-pentanedioic acid 1-(3,5-bis-trifluoromethyl-benzyl)ester. See, e.g., Natsugari et al., 1987.

### Synthesis 11

### 2-Oxo-pentanedioic acid 1-hexadecyl ester

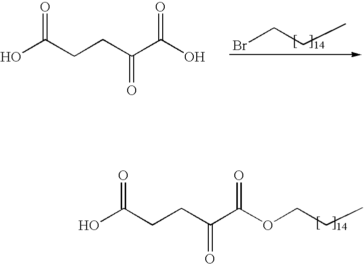

2-Oxo-pentanedioic was dissolved in THF under nitrogen. To this solution was added one equivalent of NaH. The resulting sodium salt of the α-keto acid was then treated dropwise with one equivalent of 1-hexadecyl bromide over a period of 100 hours. See, e.g., Domagala, 1980.

### Synthesis 12

### 2-Oxo-pentanedioic acid 1-tetradecyl ester

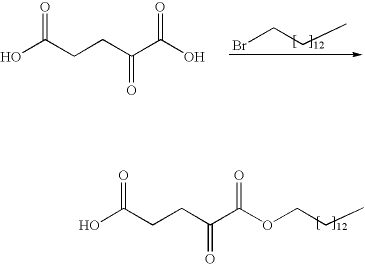

2-Oxo-pentanedioic was dissolved in THF under nitrogen. To this solution was added one equivalent of NaH. The resulting sodium salt of the α-keto acid was then treated dropwise with one equivalent of 1-tetradecyl bromide over a period of 100 hours. See, e.g., Domagala, 1980.

### Biological Methods

Plasmids. The SDHD siRNA hairpins were cloned into pBABE-Puro ΔLTR together with the U6 promoter, as previously described (Fox et al., 2003). A PCR-based strategy was employed using pEF6-hU6 as a template and primers containing SDHD DNA: hairpin sequences (Di3=sense 5′-GGTCAGACCTGCTCATATCTCAGCA-3′; Di4 sense 5′-GGTGTGGAGTGCAGCACATACAC-3′). scRNAi was cloned into pSuperRetro (OligoEngine) with the hairpin sequence, sense, 5′-GATACGGTAGGGCGACAA-3′. The scrambled and the SDHD-targeting siRNAs short hairpins, Sc and Di3, were also cloned into pSUPER-GFP-neo.

pGL2/HRE-Luciferase was generated by inserting 3 copies in tandem of the 24-mer oligonucleotide (5′-tgtcacgtcctgcacgactctagt-3′) in front of a minimal thymidine kinase promoter into pGL2-basic (Promega). The oligonucleotide contains 18 bp from PGK promoter including the HRE.

pRK5/HA-ODDD was generated by cloning the human HIF-1α ODDD (amino acids 530-652) in frame into a vector containing HA-tag and Gal4 DNA binding domain. Then the entire cDNA was cloned into pRK5 vector.

pEGFP/ODDD, the plasmid encoding the GFP-ODDD fusion protein, was generated as follows: A PCR fragment of hHIF-1α ODDD was generated using PRK5/HA-ODDD as a template and ligated in frame into pEGFP-C1 (Clontech).

pRC-CMV/HA-pVHL was used to co-express HA-pVHL in the GFP-ODD-expressing clones

Cell culture. GFP-ODD expressing clones were generated by co-transfecting pEGFP/ODD, pRC-CMV/HA-pVHL and pBabe-Puro into HEK293 cells. Following selection in puromycin, 96 clones were randomly picked and duplicated in 96-well plates each incubated with or without CoCl₂. Clones that showed low basal level of GFP fluorescence with a significant induction of fluorescence when treated with CoCl₂ were further studied.

In vitro PHD activity. HA-ODDD was in vitro-translated (IVT) using wheat germ extract (Promega) and 4 μL aliquots of the IVT reaction were incubated with 50 μL of HEK293 or HeLa cellular extracts [20 mM Tris (pH 7.4), 5 mM KCl, 1.5 mM MgCl₂, 1 mM DTT, supplemented with “complete” protease inhibitor cocktail (Roche) and 100 μM ALLN]. The reaction was carried out for 15 minutes at 37° C. in the presence of 5 mM ascorbate and 100 μM FeCl₂ with either 5 mM DFO or the indicated amounts of succinate or free α-ketoglutaric acid or α-ketoglutarate esters. Reactions were terminated by adding Laemmli sample buffer and immediate boiling. Following SDS-PAGE, samples were analyzed by western blot using an anti-HA antibody.

SDH activity (complex II; succinate—DCIP oxidoreductase). Cells were lysed with 0.1% v/v Triton ×100 in an assay buffer composed of 25 mM KHPO4 (pH 7.4), 20 mM succinate, 50 μM decylubiquinone, 5 μM rotenone, 2 μM antimycin A and 10 mM NaN₃. Following a 15 minute incubation at room temperature, the baseline absorbance at 600 nm was recorded and the reaction initiated by adding 50 μM DCIP (E=21 mM⁻¹ cm⁻¹). The change in absorbance was monitored for 2-3 minutes before and after addition of 50 μM 2-thenolytrifluoroacetone (TTFA), a complex II inhibitor used to confirm reaction specificity. The specific activity of TTFA-sensitive succinate-DCIP oxidoreductase is reported as nmol/min/mg cell protein.

Succinate quantification by GCMS. Cell extracts were prepared in 90% methanol, 10% acetic acid containing 1 μg/mL (D₄)-succinic acid. Extracts were evaporated to dryness under nitrogen at 60° C. and methylated by redissolving in 1% HCl in methanol for 10 minutes at 60° C. The extracts were re-dried and dissolved in hexane and 1 μL was injected into an Automass Multi GCMS system. The instrument was fitted with a ZB-1 column (30 metres×0.32 mm id×1 μm film), the oven was programmed as follows: 80° C. (5 minutes) then 10° C./min to 170° C.; the head pressure was 60 kPa. The mass spectrometer was operated in EI mode at 70 eV and selected ion monitoring was carried out for ions at 119 and 91 amu (methyl ester of (D₄)-succinic acid) and 115 and 87 amu (methyl ester of succinic acid).

Protein analyses. For HIF-1α immunoblot, cells were extracted in Laemmli sample buffer and for all other analyses in Tris lysis buffer [50 mM Tris (pH 8.0), 150 mM NaCl, 0.5% NP40, supplemented with “complete” protease inhibitor cocktail (Roche) and 100 μM ALLN]. Following SDS-PAGE, proteins were blotted onto nitrocellulose and analyzed with the following antibodies: anti-HIF-1α (BD Biosciences), anti-HA (Roche), anti-GFP (BD Biosciences) or anti-Actin (Sigma). Immunoprecipitation for HA-pVHL was carried out using an anti-HA antibody (Roche) and protein G sepharose beads (Phramacia) in Tris lysis buffer. Proteins were eluted by incubating the beads with 1 mg/mL HA-peptide in TBS for 15 minutes at 37° C. Eluates were immediately subjected to fluorometric analysis in a 96-well plate fluorometer (Molecular Devices) to determine GFP or GFP-ODDD levels.

Preparation of Ha-pVHL protein as a probe for far-western blot analysis was carried out as follows: pRc-CMV/HA-pVHL was transfected into HEK293 cells and 30 hours later, HA-pVHL protein was immunopurified from the cell extract on an anti-HA matrix (Roche) according to manufacture instructions. Following elution of the protein with HA peptide the eluate was dialysed over night in TBS and HA-pVHL levels were assessed by SDS-PAGE followed by Coomassie blue staining and western blot analysis.

For far-western blot analysis, protein extracts from GFP-ODDD expressing cells were blotted onto nitrocellulose membrane and blocked in 5% non-fat dry milk in TBST for 2 hours. Following several washes with TBST, HA-pVHL protein in TBST/milk (1 μg/mL) was added to the nitrocellulose membrane and incubated over night at 4° C., followed by several washes with TBST and detection with an anti-HA antibody.

Measurement of α-ketoglutarate. HEK293 cells were grown under indicated conditions. All operations were performed at 4° C. Cell monolayers were washed with PBS and lysed with RIPA buffer. Cells were collected from the plate, vortexed vigorously and the extracts centrifuged for 5 minutes at 15,000×g at 4° C. Aliquots of the extracts were analyzed immediately for α-ketoglutarate. The assay consisted of the following: 100 mM KH₂PO₄ (pH 7.2), 10 mM NH₄Cl, 5 mM MgCl₂, and 0.15 mM NADH. Following equilibration at 37° C. with extract, the reaction was started by addition of 5 units of glutamate dehydrogenase. Absorbance decrease was monitored at 340 nm. The intracellular concentration of α-ketoglutarate was determined from the absbrbance decrease in NADH (e=6.22 mM⁻¹cm⁻¹) and the packed cell volume determined from duplicate unextracted cell samples.

Spheroid Culture and Treatment. HCT116 cells were cultured in 0.5% low melting point agarose in DMEM supplemented with 10% fetal calf serum. 10 cm dishes were seeded with 20 mL of cells at 10⁵/mL for one week with a change in medium every 2 days. Cells were then harvested from five 10 cm dishes, put into a 500 mL spinner flask (60 rpm) containing 200 mL DMEM supplemented with 10% fetal calf serum and cultured for approximately 2 weeks until the average diameter was 500 μm. On the day of the experiment, 0.5 mL of spheroid suspension was then seeded into each well of a 24 well plate containing 1 mL 0.5% low melting point agarose. These spheroids were treated for 24 hours at 37° C. with either 1 mM free (underivatized) a ketoglutaric acid, 1 mM octyl-α-ketoglutarate, or 1 mM TFMB-α-ketoglutarate.

### Biological Data

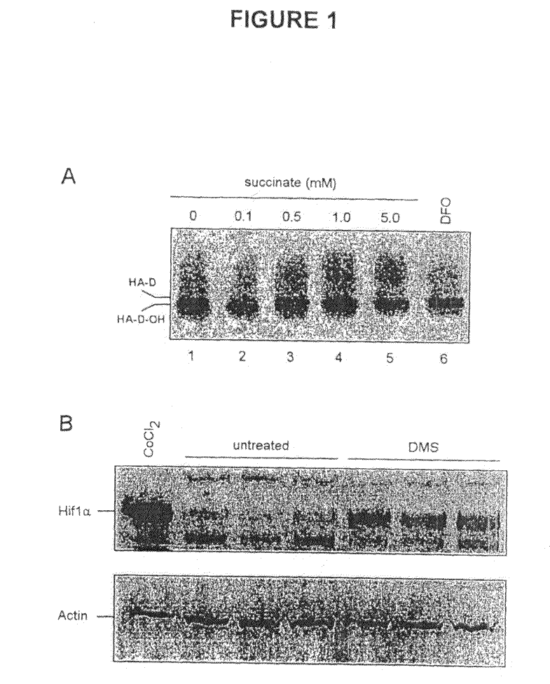

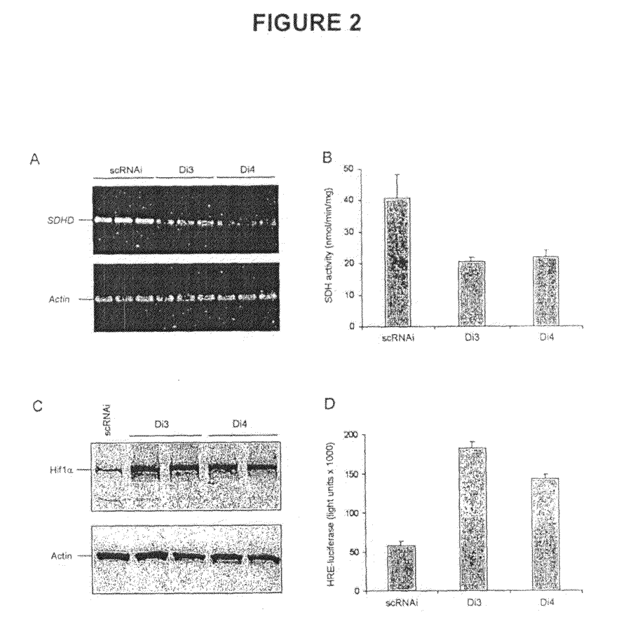

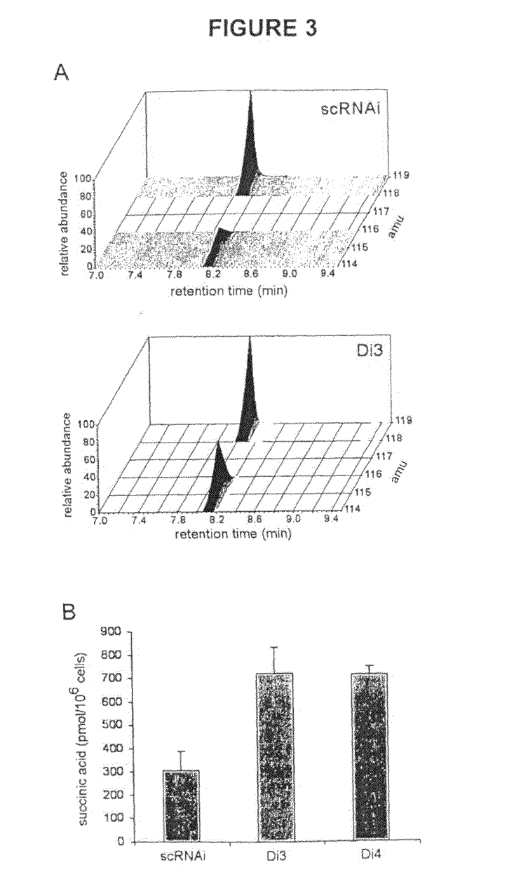

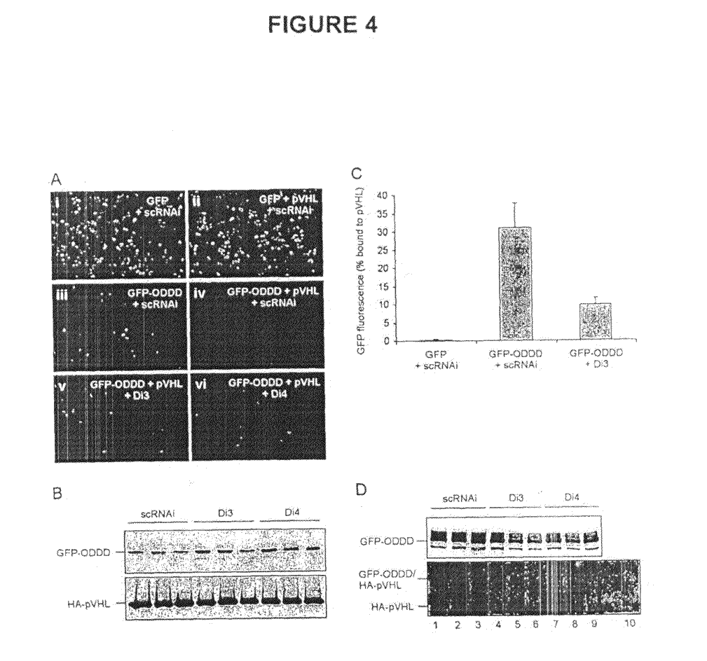

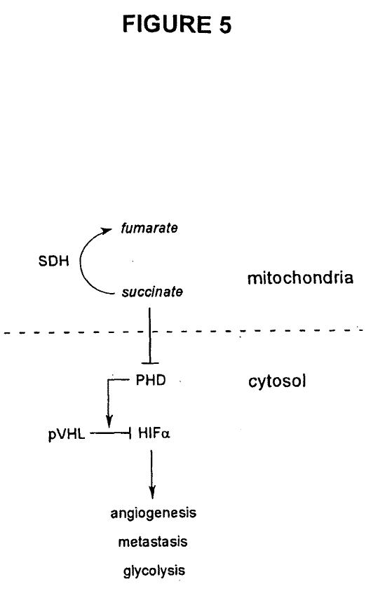

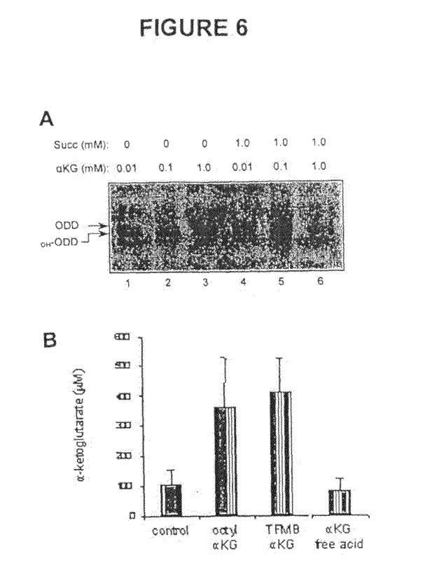

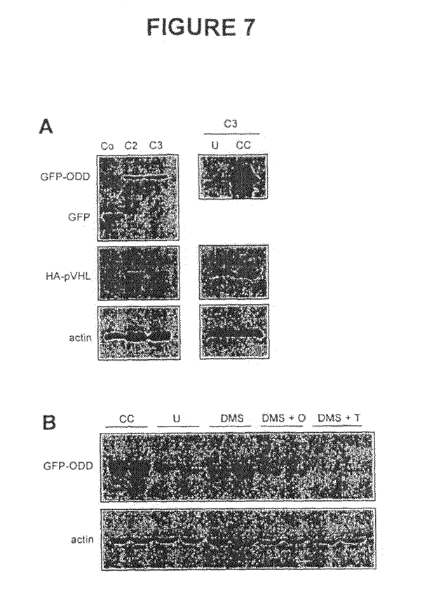

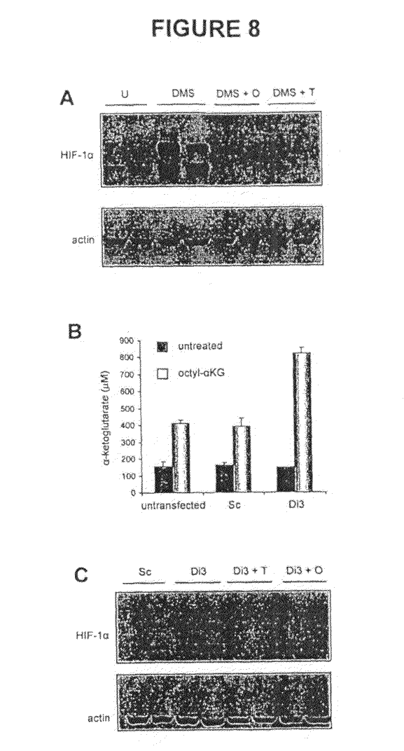

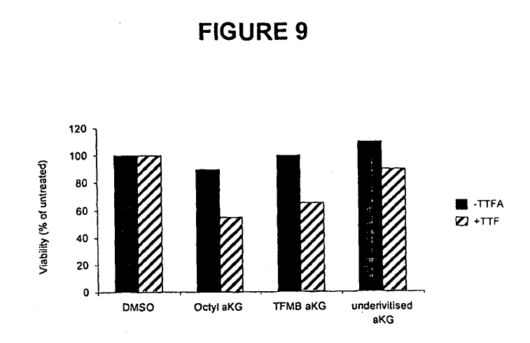

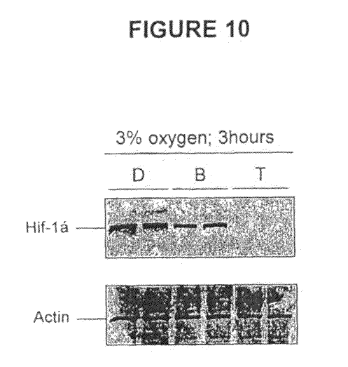

PHD activity was analysed, in vitro, in the presence of increasing amounts of succinate, in order to demonstrate that mutations in SDH result in the accumulation of succinate that then inhibits the HIFα hydroxylases in the cytosol by product inhibition. In vitro-translated HA-tagged ODDD was used as a substrate and cell extracts were used as a source of PHD activity. When the HA-ODDD was incubated with cell extracts in the presence of α-ketoglutarate, Fe²⁺, and ascorbate, it undergoes hydroxylation and migrates faster on SDS-PAGE (Ivan et al., 2001; Huang et al., 2002). Deferoxamine (DFO) is an iron chelator that inhibits PHD activity and can therefore be used as a hypoxia mimetic compound to stabilize HIF-α levels in certain cells (Safran et al., 2003). DFO inhibited PHD activity in vitro and retarded HA-ODDD mobility on SDS-PAGE. See FIG. 1A, lane 6. Increasing amounts of succinate led to a decrease in the production of hydroxylated HA-ODDD. See FIG. 1A, lanes 1-5. The IC₅₀ was calculated to be 0.5 mM for succinate under these reaction conditions.

In order to determine if high levels of succinate are sufficient to elevate HIF-1α-levels in cells, cells were incubated with the membrane-permeable dimethyl-ester succinic acid derivative (DMS) and analyzed HIF-1α levels. Although the rate of DMS uptake, conversion into succinate, and metabolism are unknown, cells incubated with 20 mM DMS for 48 hours showed elevated levels of HIF-1α under normoxic conditions. See FIG. 1B. CoCl₂, a hypoxia mimetic compound that has also been shown to inhibit PHD activity (Safran et al., 2003) was used as a positive control. See FIG. 1B.

In order to study the effect of SDH inhibition on succinate levels and PHD activity in cells, RNA interference (RNAi) was used to target SDH. Vectors encoding small interference RNA (siRNA) that target the SDHD subunit (Di3 or Di4) were constructed and their function was analyzed by transient transfection into human embryonic kidney cells (HEK293). SDHD mRNA levels were analysed by RT-PCR and found to be significantly lower in cells transfected with either of the two SDHD siRNA constructs (Di3 or Di4) as compared to scrambled siRNA (scRNAi)-transfected cells. See FIG. 2A.

In order to analyse the efficacy of siRNA to inhibit endogenous SDH activity, succinate and 2,6-dichloroindophenol (DCIP) were used as electron donor and acceptor, respectively, to spectrophotometrically measure SDH activity (Fox et al., 2003). When cells were transfected with either Di3 or Di4, SDH activity was decreased by approximately 50% in the overall cell population, as compared to scRNAi-transfected cells. See FIG. 2B. Transfection efficiency was estimated to be approximately 50% by co-transfection with a GFP-encoding plasmid (data not shown). This suggests that SDH activity in transfected cells is severely decreased at the time of analysis. Transfection of any of the siRNAs had no effect on citrate synthase activity, an unrelated nuclear-encoded TCA cycle enzyme (data not shown). The effect of SDH inhibition on HIF-1α levels was analysed by western blot. A clear induction of HIF-1α protein under normoxic conditions is observed in Di3- and Di4-transfected cells as compared to the control-transfected cells. See FIG. 2C. Moreover, when HIF activity was analyzed by co-transfection of a vector containing luciferase reporter gene downstream to a minimal promoter with a HIF responsive element (HRE), a three-fold induction in HIF activity was observed in the SDH-inhibited cell population. See FIG. 2D.

In order to determine if the observed SDH inhibition led to an accumulation of succinate, gas chromatography mass spectrometry (GCMS) was used to measure succinate levels in cells following siRNA transfection. The amount of succinic acid in cell extracts was calculated by comparison to a known amount of deuterated (D₄)-succinic acid that was used as a reference in the lysis solution. See FIG. 3A. When cells were transfected with either of the SDHD-targeting siRNAs, an approximately 2.5 fold increase in succinic acid was observed. See FIG. 3A and FIG. 3B. Based on approximately 50% transfection efficiency, the levels of succinate in the transfected cells are under-estimated.

In summary, these results provide a direct link between SDH inhibition and the consequent accumulation of succinate with elevated HIF-1α protein levels.

Next, it was determined whether or not elevated succinate levels following SDH inhibition decreased PHD activity in cells. A GFP-ODDD fusion protein was used, the degradation of which is enhanced by pVHL over-expression (see FIG. 4A, (iii) and (iv)) and therefore depends on ODDD hydroxylation for degradation, in contrast to GFP (see FIG. 4A, (i) and (ii)). When cells were co-transfected with GFP-ODDD, HA-tagged pVHL and either of the siRNA constructs, higher GFP levels were observed in Di3- and Di4-transfected cells compared to scRNAi-transfected cells. See FIG. 4; (iv), (v), and (vi).

In order to quantify the differences in GFP-ODDD levels, cells were lysed and the amounts of GFP-ODDD in the extracts were determined by western blot using an anti-GFP antibody. See FIG. 4B, upper panel. The differences in GFP-ODDD levels could not be attributed to differences in pVHL levels in the transfected cells since HA-pVHL was equally expressed in each of the transfections. See FIG. 4B, lower panel. Therefore, it is likely that in SDH-inhibited cells, the HIF-1α ODDD is hydroxylated less efficiently and consequently the binding of pVHL to GFP-ODDD is reduced.

In order to test this possibility, cells were transfected with HA-pVHL, either scRNAi or Di3 and with GFP-ODDD or GFP (the latter were used as negative control). GFP fluorescence was analyzed in extracts prior to, or after, immunoprecipitation with an anti-HA antibody, to measure both total and VHL-bound GFP. Following stringent washes, immunoprecipitated proteins were eluted using HA peptide and GFP fluorescence was analyzed (VHL-bound GFP) and compared to that of extracts prior to immunoprecipitation (total GFP). The results are presented in FIG. 4C as percent VHL-bound GFP of total GFP.

Although GFP-transfected cells have much higher-total fluorescence than GFP-ODDD-transfected cells (see FIG. 4A, (ii) and (iv)), VHL-associated GFP fluorescence was hardly detectable, demonstrating that in the GFP-ODDD transfected cells, pVHL-associated GFP fluorescence is ODDD-dependent. In Di3-transfected cells, even though the total GFP-ODDD was higher than in scRNAi-transfected cells (see FIG. 4A, (iv) and (v)), the levels of pVHL-associated fluorescence were significantly lower (see FIG. 4C). Since pVHL binding to the HIF-1α ODDD is dependent on PHD activity (Kaelin et al., 2002; Safran et al., 2003), these results indicate that inhibiting SDH activity in cells results in decreased PHD activity.

Additionally, cells were transfected with GFP-ODDD and either scRNAi, Di3 or Di4. The hydroxylation status of GFP-ODDD was determined by examining the binding of purified HA-pVHL protein to GFP-ODDD expressed in these cells by far-western blot. The samples were normalized for GFP-ODDD levels (see FIG. 4D, upper panel) and the nitrocellulose membrane was probed with purified HA-pVHL protein. GFP-ODDD/HA-pVHL complexes were detected by an anti-HA antibody. See FIG. 4D, lower panel. HA-pVHL binding to the GFP-ODDD extracted from scRNAi transfected cells was observed (see FIG. 4D, lanes 1-3), but a significant decrease in HA-pVHL binding to the same quantity of GFP-ODDD was evident in Di3 and Di4 transfected cells (see FIG. 4D, lanes 4-9). These results suggest that PHD activity is reduced in SDH-inhibited cells.

These results provide clear evidence for a previously unidentified mechanism of regulating PHD activity and consequently HIF-1α levels, specifically, a direct signalling pathway that links mitochondrial dysfunction to tumorigenesis. These results show that succinate may function as an intracellular messenger between mitochondria and the cytosol and has a profound effect on cytosolic enzymes and consequently on nuclear events (i.e., gene expression) (see FIG. 5): Succinate is the substrate for SDH in the mitochondria and is a product of PHD activity in the cytosol where α-ketoglutarate is converted to succinate. Thus, a mechanistic link has been shown between mutations in SDH and the highly vascularized tumours which develop as a consequence of HIF-1α induction in the absence of VHL mutations.

On the basis of these results, it was postulated that increasing the levels of α-ketoglutarate in cells may facilitate the activity of PHD and serve to lower HIF-1α levels in cells in which HIF-1α is stabilized and activated due to interference with PHD activity.

The effect of α-ketoglutarate on PHD activity was examined by employing an in vitro hydroxylation assay. In this assay, hydroxylated and non-hydroxylated species are resolved using a gel-shift migration assay. PHD activity is determined using in vitro translated HA-tagged ODD as substrate and HeLa cell extracts as a source of PHD. When HA-ODD was incubated with cell extracts in the presence of Fe²⁺ and ascorbate and in the absence of succinate, even low concentrations of α-ketoglutarate led to near maximal hydroxylation of HA-ODD, as evident from its faster migration on SDS-PAGE (see FIG. 6A, lanes 1-3). When succinate concentration was held constant at 1 mM, inhibition of PHD activity was evident by the appearance of the non-hydroxylated, slower migrating form of HA-ODD (see FIG. 6A, lane 4). Addition of increasing amounts of α-ketoglutarate ranging from 0.1 to 1 mM elicited a concentration-dependent stimulation of PHD activity and progressively led to increased production of the faster migrating, hydroxylated form of HA-ODD (see FIG. 6A, lanes 4-6). These results demonstrate that succinate competitively inhibits PHD and that α-ketoglutarate can reverse this PHD inhibition in vitro.

In order to investigate this hypothesis, several membrane permeable α-ketoglutarates (i.e., esters) were synthesised and their effect on PHD activity analyzed, that is, their effect on HIF-1α levels in cells with defects in TCA cycle enzymes such as reduced SDH activity or cells under hypoxic conditions that are known to induce high levels of HIF-1α.

Free α-ketoglutaric acid is hydrophilic and cannot efficiently cross the plasma membrane to reach sufficiently high intracellular levels. Membrane-permeable monoester derivatives of α-ketoglutaric acid with different hydrophobic indices were designed and synthesized. Once these derivatives enter the cells, the ester is hydrolyzed by cytosolic esterases, increasing the concentration of α-ketoglutarate in the cytosol. In the cytosol, the free acid exists predominantly as an anion which is referred to as α-ketoglutarate. With efficient plasma membrane permeability and equilibration, [α-ketoglutarate-ester]ᵢₙ should equal [α-ketoglutarate-ester]ₒᵤₜ. The conversion of α-ketoglutarate ester to α-ketoglutaric acid by cytosolic esterases both traps α-ketoglutarate in the cell and creates a concentration gradient to drive more α-ketoglutarate ester from the medium into the cytosol. The α-ketoglutarate thus formed and metabolized in the cells would be rapidly replaced by exogenous α-ketoglutarate ester.

In order to examine the ability of α-ketoglutarate esters to increase cellular levels of α-ketoglutarate, HEK293 cells were incubated with 1 mM of either free α-ketoglutaric acid, octyl-α-ketoglutarate, or trifluoromethyl benzyl (TFMB)-α-ketoglutarate for 5 hours. Cell extracts were prepared and immediately analyzed for intracellular α-ketoglutarate levels using a spectrophotometric assay that employs glutamate dehydrogenase (GDH). High levels of NH₄⁺ and NADH were used to shift the GDH reaction toward glutamate formation (reductive amination), in which the concentration of α-ketoglutaric acid is stoichiometric to the amount of NADH oxidized. Residual non-hydrolyzed α-ketoglutarate's esters present in cells at the time of extraction do not contribute to the measurement of the free acid.

When cells were treated with octyl-α-ketoglutarate or TFMB-α-ketoglutarate for 5 hours prior to extraction, α-ketoglutarate in cells rose from a basal (endogenous) level of approximately 100 μM to 350-400 μM (see FIG. 6B). However, cells treated with free α-ketoglutaric acid showed no increase in the level of intracellular α-ketoglutarate above basal levels (see FIG. 6B). These results show that uptake and hydrolysis of exogenously added octyl- and TFMB-α-ketoglutarate esters lead to a significant elevation of intracellular α-ketoglutarate.

In order to analyze the effect of α-ketoglutarates on PHD activity in cells, two cell lines expressing both a GFP-ODD fusion protein and pVHL (see FIG. 7A left) were generated. In these cells, GFP levels are tightly regulated by PHD activity, which targets the GFP-ODD fusion protein for pVHL-mediated proteasomal degradation. When the cells were treated with CoCl₂, a hypoxia-mimetic compound that inhibits PHD activity, an increase in GFP-ODD levels was observed by western blot analyses (see FIG. 7A right). Hence, GFP fluorescence levels in these cells report the activity of PHD. To determine whether increased levels of intracellular α-ketoglutarate can overcome succinate-mediated PHD inhibition, cells were first treated with dimethyl succinate (DMS). 48 hours after treatment with DMS, an increase in GFP fluorescence was observed. It is important to note that in order to achieve maximal induction of GFP-ODD with DMS, cells were maintained at 10% oxygen. This level of oxygen has no effect on basal levels of GFP-ODD. Within 12 hours of addition to DMS-treated cells, octyl-α-ketoglutarate or TFMB-A-ketoglutarate reduced GFP fluorescence, demonstrating a stimulation of PHD activity. Western blots corresponding to these treatments (see FIG. 7B) showed that the level of GFP-ODD rose when cells were treated with DMS and fell to the basal level when the α-ketoglutarate esters were added.

The ability of α-ketoglutarate to destabilize HIF1α in HEK293 cells in which HIF1α was induced by DMS was studied next. Cells were incubated with DMS for 24 hours after which either octyl-α-ketoglutarate or TFMB-α-ketoglutarate was added, followed by 3 hours of incubation and then cell lysis and analysis of HIF1α levels. Treatment with the TFMB ester did substantially reduce the levels of HIF1α protein, while the octyl ester reversed DMS-induction of HIF1α completely (see FIG. 8A).

It is important to note that, in the above experiments, cells treated with free α-ketoglutaric acid and α-ketoglutarate esters have a functional TCA cycle. The inventors hypothesized that in cells with a dysfunctional TCA cycle, in which metabolism of TCA cycle intermediates is impaired, larger amounts of α-ketoglutarate will accumulate. In order to test this hypothesis, cells were transiently transfected with the Di3 shRNA, targeting the SDHD subunit and known to efficiently suppress SDH activity in cells. Scrambled shRNA (Sc) was used for negative control. 48 hours after transfection, cells were either left untreated or treated with 1 mM octyl-α-ketoglutarate for 5 hours after which intracellular α-ketoglutarate levels were analyzed as described above. None of the shRNA constructs had an effect on the level of basal α-ketoglutarate of untreated cells. However, SDH-deficient cells that were treated with octyl-α-ketoglutarate showed a two-fold rise in α-ketoglutarate levels above the rise observed in the scrambled control-transfected cells (see FIG. 8B).

In order to determine whether increasing the concentration of α-ketoglutarate in SDH-inhibited cells will counteract HIF1α induction, cells were transfected with Di3 shRNA and treated with α-ketoglutarate esters. The HIF1α induction observed with Di3 transfection was reversed by a 24 hour treatment with either octyl-α-ketoglutarate or TFMB-α-ketoglutarate (see FIG. 8C). This demonstrates that PHD inhibition, when caused by SDH dysfunction and the consequent rise in succinate, can be overcome by excess α-ketoglutarate.

Cells were also grown continuously in the presence or absence of a succinate dehydrogenase (SDH) inhibitor—(TTFA) in a medium that can sustain cells with dysfunctional oxidative phosphorylation (with excess of pyruvate and uridine). Where indicated, cells were either treated with vehicle control (DMSO), free α-ketoglutaric acid control (αKG) or with TFMB-α-ketoglutarate (T-αKG). Only the treatment that lead to increase intracellular α-ketoglutarate (T-αKG) in cells with dysfunctional SDH activity lead to a significant death (see FIG. 9).

These studies support a mitochondria-to-cytosol signaling mechanism, by which TCA cycle impairment promotes tumorigenesis. These results demonstrate that an excess of α-ketoglutarate, a substrate for PHD, overcomes PHD inactivation. Succinate inhibition of PHD is competitive in nature. Therefore, the ratio (rather than the absolute concentrations) of α-ketoglutarate to succinate in cells will critically affect PHD activity and HIF1α stability. In order to counter the PHD-inhibiting effect of succinate, cell-permeable α-ketoglutarate esters were designed, which when hydrolyzed in the cytosol, support PHD activity thereby lowering HIF1α levels in SDH deficient cells. The inventors have shown that α-ketoglutarate preferentially accumulates in SDH deficient cells, probably because TCA cycle metabolism is impaired. This ensures that upon treatment with readily permeating drugs, the concentration of α-ketoglutarate in target cells will rise to a level fully capable of countering the effect of succinate. Our study suggests that well designed α-ketoglutarate esters have therapeutic potential in the treatment of tumours with functional down-regulation or mutations of SDH, where it could restore normal low levels of HIF1α.

In order to investigate this hypothesis in cells under hypoxic conditions, the effect of the membrane permeable α-ketoglutarate esters on HIF-1α levels hypoxic cells was also analyzed.

Initial analysis with two compounds (α-ketoglutarate benzyl ester; α-ketoglutarate trifluoromethylbenzyl ester) led to partial or complete recovery of normal HIF-1α levels in cells under hypoxia (3% oxygen) (see FIG. 10). Cells were also grown under hypoxic conditions (0.5% oxygen), in the presence of either free α-ketoglutaric acid or octyl-αKG. Only conditions that lead to increase intracellular α-ketoglutarate level (treatment with Octyl-αKG) resulted in significant cell death.

Epithelial cells were grown on semi-solid medium (matrigel) preventing them from adhering to the plate. Under these conditions, cells form spheres that can grow to a few millimetres in diameter. While growing, these spheres develop a necrotic zone in the centre, very much like most solid tumours. This is due to limitations in oxygen and nutrients diffusion. Cells were grown for several days under these conditions and then treated with either free α-ketoglutaric acid or TFMB-αKG or Octyl-αKG esters. Only treatment that led to increase in intracellular α-ketoglutarate levels (TFMB-αKG or Octyl-αKG) led to the inability of cells to sustain growth in three dimensions.

It is likely that, apart from HIFα, there may be other substrates for PHD that may contribute to the tumorigenic effect of succinate. Moreover, PHDs are not the only α-ketoglutarate-dependent hydroxylases in cells. Therefore, succinate accumulation in SDH-deficient tumours may have a more far-reaching effect on tumour development, leading to a wide variety of possible biochemical outcomes that link mitochondrial dysfunction to tumorigenesis. Whatever other enzymes or substrates might be regulated by succinate, it is likely that increasing α-ketoglutarate levels in cells will reverse this effect and potentially block tumour maintenance.

The foregoing has described the principles, preferred embodiments, and modes of operation of the present invention. However, the invention should not be construed as limited to the particular embodiments discussed. Instead, the above-described embodiments should be regarded as illustrative rather than restrictive, and it should be appreciated that variations may be made in those embodiments by workers skilled in the art without departing from the scope of the present invention.

The present invention is not limited to those embodiments which are encompassed by the appended claims, which claims pertain to only some of many preferred groups of embodiments, and which claims are included at this time primarily for initial search purposes.

### REFERENCES

A number of patents and publications are cited above in order to more fully describe and disclose the invention and the state of the art to which the invention pertains. Full citations for these references are provided below. Each of these references is incorporated herein by reference in its entirety into the present disclosure, to the same extent as if each individual reference was specifically and individually indicated to be incorporated by reference.
Albayrak, T., Scherhammer, V., Schoenfeld, N., Braziulis, E., Mund, T., Bauer, M. K., Scheffler, I. E., and Grimm, S., 2003, “The tumor suppressor cybL, a component of the respiratory chain, mediates apoptosis induction,” Mol. Biol. Cell., Vol. 14, pp. 3082-3096.
Baysal, B. E., 2003, “On the association of succinate dehydrogenase mutations with hereditary paraganglioma,” Trends Endocrinol. Metab., Vol. 14, pp. 453-459.
Baysal, B. E., et al., 2000, Science, Vol. 287, p. 848.
Beyerman, H. C., et al., 1961, BSCFAS, Bull. Soc. Chim. Fr., pp. 1812-1820.
Bruick, R. K. McKnight, S. L., 2001, Science, Vol. 294, p. 1337.
Corey, E. J., Erickson, B. W., 1971, J. Org. Chem., p. 3553.
Covello, K. L. and Simon, M. C., 2004, “HIFs, hypoxia, and vascular development,” Curr. Top. Dev. Biol., Vol. 62, pp. 37-54.
Dolmans, D. E., Fukumura, D., Jain, R. K., 2003, Nat. Rev. Cancer, Vol. 3, p. 380.
Domagala, J. M., 1980, Tetrahedron Letters, p. 4997.
Domagala, John M., 1980, Tetrahedron Letters, Vol. 21, pp. 4997-5000
Eliel, E. L., Hartmann, A. A., 1978, J. Org. Chem., p. 505.
Eng, C., Kiuru, M., Fernandez, M. J., and Aaltonen, L. A., 2003, “A role for mitochondrial enzymes in inherited neoplasia and beyond,” Nat. Rev. Cancer, Vol. 3, pp. 193-202.
Epstein, A. C., et al., 2001, Cell, Vol. 107, p. 43.
Fox, C. J., et al., 2003, Genes Dev., Vol. 17, p. 1841.
Frederiksen, C. M., Knudsen, S., Laurberg, S., Orntoft, T. F., 2003, J. Cancer Res. Clin. Oncol., Vol. 129, p. 263.
Gerald, D., Berra, E., Frapart, Y. M., Chan, D. A., Giaccia, A. J., Mansuy, D., Pouyssegur, J., Yaniv, M., and Mechta-Grigoriou, F., 2004, “JunD reduces tumor angiogenesis by protecting cells from oxidative stress,” Cell, Vol. 118, pp. 781-794.
Gimenez-Roqueplo, A. P., Favier, J., Rustin, P., Mourad, J. J., Plouin, P. F., Corvol, P., Rotig, A., and Jeunemaitre, X., 2001, “The R22X mutation of the SDHD gene in hereditary paraganglioma abolishes the enzymatic activity of complex II in the mitochondrial respiratory chain and activates the hypoxia pathway,” Am. J. Hum. Genet., Vol. 69, pp. 1186-1197.
Gimenez-Roqueplo, A. P., Favier, J., Rustin, P., Rieubland, C., Kerlan, V., Plouin, P. F., Rotig, A., and Jeunemaitre, X., 2002, “Functional consequences of a SDHB gene mutation in an apparently sporadic pheochromocytoma,” J. Clin. Endocrinol. Metab., Vol. 87, pp. 4771-4774.
Habano, W., et al., 2003, Oncol. Rep., Vol. 10, p. 1375.
Hartenstein, H., et al., 1993, JPCCEM. J. Prakt. Chem./Chem.-Ztq., Vol. 335, No. 2, pp. 176-180.
Hewitson, K. S., et al., 2002, J. Biol. Chem., Vol. 277, p. 26351.
Huang, J., Zhao, Q., Mooney, S. M., Lee, F. S., 2002, “Sequence determinants in hypoxia-inducible factor-1alpha for hydroxylation by the prolyl hydroxylases PHD1, PHD2, and PHD3,” J. Biol. Chem., Vol. 277, pp. 39792-39800.
Ishii, T., Yasuda, K., Akatsuka, A., Hino, O., Hartman, P. S., and Ishii, N., 2005, “A mutation in the SDHC gene of complex II increases oxidative stress, resulting in apoptosis and tumorigenesis,” Cancer Res., Vol. 65, pp. 203-209.
Ivan, M. et al., 2001, Science, Vol. 292, p. 464.
Ivan, M., et al., 2002, Proc. Natl. Acad. Sci. USA, Vol. 99, p. 13459.
Jaakkola, P., et al., 2001, Science, Vol. 292, p. 468.
Kaelin, W. G., Jr., 2002, Nat. Rev. Cancer, Vol. 2, p. 673.
Kim, W. Y., and Kaelin, W. G., 2004, “Role of VHL gene mutation in human cancer,” J. Clin. Oncol., Vol. 22, pp. 4991-5004.
Lando, D., et al., 2002b, Genes Dev., Vol. 16, p. 1466.
Lando, D., Peet, D. J., Whelan, D. A., Gorman, J. J., Whitelaw, M. L., 2002a, Science, Vol. 295, p. 858.
Le Boucher, J., Obled, C., Farges, M. C., Cynober, L., 1997, “Ornithine alpha-ketoglutarate modulates tissue protein metabolism in burn-injured rats,” Am. J. Physiol., Vol. 273 (3 Pt 1), pp. E557-63.
Messner, K. R., and Imlay, J. A., 2002, “Mechanism of superoxide and hydrogen peroxide formation by fumarate reductase, succinate dehydrogenase, and aspartate oxidase,” J. Biol. Chem., Vol. 277, pp. 42563-42571.
Natsugari, H., et al., 1987, J. Chem. Soc. Chem. Commun., p. 62.
Natsugari, Hideaki, et al., 1987, J. Chem. Soc. Chem. Commun., Vol. 2, pp. 62-63.
Olah, G. A., et al., 1991, Synthesis, p. 204.
Pennacchietti, S., et al., 2003, Cancer Cell, Vol. 3, p. 347.
Pollard, P. J., Briere, J. J., Alam, N. A., Barwell, J., Barclay, E., Wortham, N. C., Hunt, T., Mitchell, M., Olpin, S., Moat, S. J., et al., 2005, “Accumulation of Krebs cycle intermediates and over-expression of HIF1α in tumours which result from germline FH and SDH mutations,” Hum. Mol. Genet., Vol. 14, pp. 2231-2239.
Pollard, P. J., Wortham, N. C., Tomlinson, P., 2003, “The TCA cycle and tumorigenesis: the examples of fumarate hydratase and succinate dehydrogenase,” Ann. Med., Vol. 35, pp. 632-639.
Pollard, P., Wortham, N., Barclay, E., Alam, A., Elia, G., Manek, S., Poulsom, R., and Tomlinson, I., 2005, “Evidence of increased microvessel density and activation of the hypoxia pathway in tumours from the hereditary leiomyomatosis and renal cell cancer syndrome,” J. Pathol., Vol. 205, pp. 41-49.
Pugh, C. W., Ratcliffe, P. J., 2003, Nat. Med., Vol. 9, p. 677.
Safran, M. Kaelin, W. G., Jr., 2003, “HIF hydroxylation and the mammalian oxygen-sensing pathway,” J. Clin. Invest., Vol. 111, p. 779-783.
Schofield, C. J., and Ratcliffe, P. J., 2004, “Oxygen sensing by HIF hydroxylases,” Nat. Rev. Mol. Cell. Biol., Vol. 5, pp. 343-354.
Schofield, C. J., Zhang, Z., 1999, Curr. Opin. Struct. Biol., Vol. 9, p. 722.
Schwetlick, K., et al., 2001, Organikum, 21st Edition, (published by Wiley-VCH, Weinheim), p. 489.
Selak, M. A., Armour, S. M., MacKenzie, E. D., Boulahbel, H., Watson, D. G., Mansfield, K. D., Pan, Y., Simon, M. C., Thompson, C. B., and Gottlieb, E. (2005). Succinate links TCA cycle dysfunction to oncogenesis by inhibiting HIF-alpha prolyl hydroxylase. Cancer Cell 7, 77-85.
Semenza, G. L., 2002, “HIF-1 and tumor progression: pathophysiology and therapeutics,” Trends Mol. Med., Vol. 8, p. S62-S67.
Staller, P., et al., 2003, Nature, Vol. 425, p. 307.
Starkey, E. B., 1943, Organic Syntheses, Coll. Vol. 2, p. 225.
Takeuchi, Yasuo, et al., 1999, “Synthesis and siderophore activity of vibrioferrin and its diastereomeric isomer,” Chem. Pharm. Bull., Vol. 47, No. 9, pp. 1284-1287.
Trounce, I. A., Kim, Y. L., Jun, A. S., Wallace, D. C., 1996, Methods Enzymol., Vol. 264, p. 484.

Williamson, J. R., and Corkey, B. E., 1979, “Assay of citric acid cycle intermediates and related compounds—update with tissue metabolite levels and intracellular distribution,” Methods Enzymol., Vol. 55, pp. 200-222.
Yankovskaya, V., Horsefield, R., Tornroth, S., Luna-Chavez, C., Miyoshi, H., Leger, C., Byrne, B., Cecchini, G., and Iwata, S., 2003, “Architecture of succinate dehydrogenase and reactive oxygen species generation,” Science, Vol. 299, pp. 700-704.
Yu, F., White, S. B., Zhao, Q., Lee, F. S., 2001, Proc. Natl. Acad. Sci. USA, Vol. 98, p. 9630.

1-73. (canceled)

74. An α-ketoglutarate compound having a hydrophobic moiety that is, or is part of, an ester group formed from one of the acid groups of α-ketogluartic acid; and pharmaceutically acceptable salts, solvates, amides, esters, ethers, N-oxides, chemically protected forms, and prodrugs thereof.

75. A compound according to claim 74, wherein the hydrophobic moiety is derived from one of: lipids, fatty acids, phospholipids, sphingolipids, acylglycerols, waxes, sterols, steroids (e.g., cholesterol), terpenes, prostaglandins, thromboxanes, leukotrienes, isoprenoids, retenoids, biotin, and hydrophobic amino acids (e.g., tryptophan, phenylalanine, isoleucine, leucine, valine, methionine, alanine, proline, and tyrosine).

76. A compound selected from compounds having the following formula: 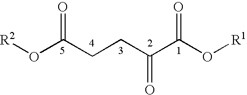
wherein each of R¹ and R² is independently selected from:
(i) H; and
(ii) a hydrophobic moiety;
with the proviso that R¹ and R² are not both H;
and pharmaceutically acceptable salts, solvates, amides, esters, ethers, N-oxides, chemically protected forms, and prodrugs thereof.

77. A compound according to claim 76, wherein neither R¹ nor R² is H.

78. A compound according to claim 76, wherein neither R¹ nor R² is H; and R¹ and R² are different.

79. A compound according to claim 76, wherein neither R¹ nor R² is H; and R¹ and R² are identical.

80. A compound according to claim 76, wherein exactly one of R¹ and R² is H.

81. A compound according to claim 76, wherein R¹ is H (and R² is not H):

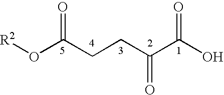

82. A compound according to claim 76, wherein R² is H (and R¹ is not H):

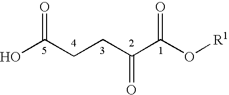

83. A compound according to claim 76, wherein the hydrophobic moiety, or each hydrophobic moiety, is independently selected from:
C₁-C₃₀ alkyl;
C₂-C₃₀ alkenyl;
C₂-C₃₀ alkynyl;
C₃-C₃₀ cycloalkyl;
C₃-C₃₀ cycloalkenyl;
C₃-C₃₀ cycloalkynyl;
C₆-C₂₀ carboaryl;
C₅-C₂₀ heteroaryl;
C₆-C₂₀ carboaryl-C₁-C₇ alkyl;
C₅-C₂₀ heteroaryl-C₁-C₇ alkyl;
and is unsubstituted or substituted;

84. A compound according to claim 76, wherein the hydrophobic moiety, or each hydrophobic moiety, is independently selected from:
C₈-C₃₀ alkyl;
C₈-C₃₀ alkenyl;
C₈-C₃₀ alkynyl;
and is unsubstituted or substituted.

85. A compound according to claim 76, wherein the hydrophobic moiety, or each hydrophobic moiety, is independently C₈-C₃₀ alkyl and is unsubstituted or substituted.

86. A compound according to claim 76, wherein the hydrophobic moiety, or each hydrophobic moiety, is independently C₄-C₂₀ alkyl and is unsubstituted or substituted.

87. A compound according to claim 76, wherein the hydrophobic moiety, or each hydrophobic moiety, is independently C₆-C₁₈ alkyl and is unsubstituted or substituted.

88. A compound according to claim 76, wherein the hydrophobic moiety, or each hydrophobic moiety, is independently C₈-C₁₆ alkyl and is unsubstituted or substituted.

89. A compound according to claim 76, wherein the hydrophobic moiety, or each hydrophobic moiety, is independently —(CH₂)ₙCH₃, wherein n is independently an integer from 5 to 29.

90. A compound according to claim 76, wherein the hydrophobic moiety, or each hydrophobic moiety, is independently —(CH₂)ₙCH₃, wherein n is independently an integer from 7 to 23.

91. A compound according to claim 76, wherein the hydrophobic moiety, or each hydrophobic moiety, is independently —(CH₂)ₙCH₃, wherein n is independently an integer from 9 to 19.

92. A compound according to claim 76, wherein the hydrophobic moiety, or each hydrophobic moiety, is independently —(CH₂)ₙCH₃, wherein n is independently an integer from 9 to 17.

93. A compound according to claim 76, wherein the hydrophobic moiety, or each hydrophobic moiety, is independently selected from:
C₆-C₂₀ carboaryl;
C₅-C₂₀ heteroaryl;
C₆-C₂₀ carboaryl-C₁-C₇ alkyl;
C₅-C₂₀ heteroaryl-C₁-C₇ alkyl;
and is unsubstituted or substituted.

94. A compound according to claim 76, wherein the hydrophobic moiety, or each hydrophobic moiety, is independently selected from:
C₆-C₁₂ carboaryl;
C₅-C₁₂ heteroaryl;
C₆-C₁₂ carboaryl-C₁-C₇ alkyl;
C₅-C₁₂ heteroaryl-C₁-C₇ alkyl;
and is unsubstituted or substituted.

95. A compound according to claim 76, wherein the hydrophobic moiety, or each hydrophobic moiety, is independently selected from:
C₆-C₁₀ carboaryl;
C₅-C₁₀ heteroaryl;
C₆-C₁₀ carboaryl-C₁-C₇ alkyl;
C₅-C₁₀ heteroaryl-C₁-C₇ alkyl;
and is unsubstituted or substituted.

96. A compound according to claim 76, wherein the hydrophobic moiety, or each hydrophobic moiety, is independently selected from:
C₆-C₂₀ carboaryl;
C₆-C₂₀ carboaryl-C₁-C₇ alkyl;
and is unsubstituted or substituted.

97. A compound according to claim 76, wherein the hydrophobic moiety, or each hydrophobic moiety, is independently selected from:
C₆-C₁₂ carboaryl;
C₆-C₁₂ carboaryl-C₁-C₇ alkyl;
and is unsubstituted or substituted.

98. A compound according to claim 76, wherein the hydrophobic moiety, or each hydrophobic moiety, is independently an optionally substituted phenyl group of formula: 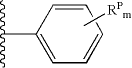
wherein m is independently 0, 1, 2, 3, 4, or 5, and each R^{P}, if present, is independently a substituent.

99. A compound according to claim 76, wherein the hydrophobic moiety, or each hydrophobic moiety, is independently an optionally substituted benzyl group of formula: 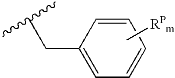
wherein m is independently 0, 1, 2, 3, 4, or 5, and each R^{P}, if present, is independently a substituent.

100. A compound according to claim 76, wherein each substituent on said hydrophobic moiety or moieties, including R^{P}, if present, is independently selected from the following:
(1) carboxylic acid; (2) ester; (3) amido or thioamido; (4) acyl; (5) halo; (6) cyano; (7) nitro; (8) hydroxy; (9) ether; (10) thiol; (11) thioether; (12) acyloxy; (13) carbamate; (14) amino; (15) acylamino or thioacylamino; (16) aminoacylamino or aminothioacylamino; (17) sulfonamino; (18) sulfonyl; (19) sulfonate; (20) sulfonamido; (21) C₅₋₂₀aryl-C₁₋₇alkyl; (22) C₆₋₂₀carboaryl and C₅₋₂₀heteroaryl; (23) C₃₋₂₀heterocyclyl; (24) C₁₋₇alkyl; C₈₋₃₀alkyl; C₂₋₇alkenyl; C₂₋₇alkynyl; C₃₋₇cycloalkyl; C₃₋₇cycloalkenyl; C₃₋₇cycloalkynyl.

101. A compound according to claim 76, wherein each substituent on said hydrophobic moiety or moieties, including R^{P}, if present, is independently selected from: halo; cyano; nitro; hydroxy; C₁-C₇alkyoxy; C₁-C₇alkyl; C₁-C₇ haloalkyl; and C₈-C₃₀ alkyl.

102. A compound according to claim 76, wherein each substituent on said hydrophobic moiety or moieties, including R^{P}, if present, is independently selected from: halo; C₁-C₄ alkyl; C₁-C₄ haloalkyl; and C₁₂-C₂₂ alkyl.

103. A compound according to claim 76, wherein each substituent on said hydrophobic moiety or moieties, including R^{P}, if present, is independently selected from: fluoro; C₁-C₄ alkyl; and C₁-C₄ fluoroalkyl.

104. A compound according to claim 76, wherein each substituent on said hydrophobic moiety or moieties, including R^{P}, if present, is independently selected from: F, —CH₃, —CF₃.

105. A compound according to claim 76, selected from the following compounds, and pharmaceutically acceptable salts, solvates, amides, esters, ethers, N-oxides, chemically protected forms, and prodrugs thereof:

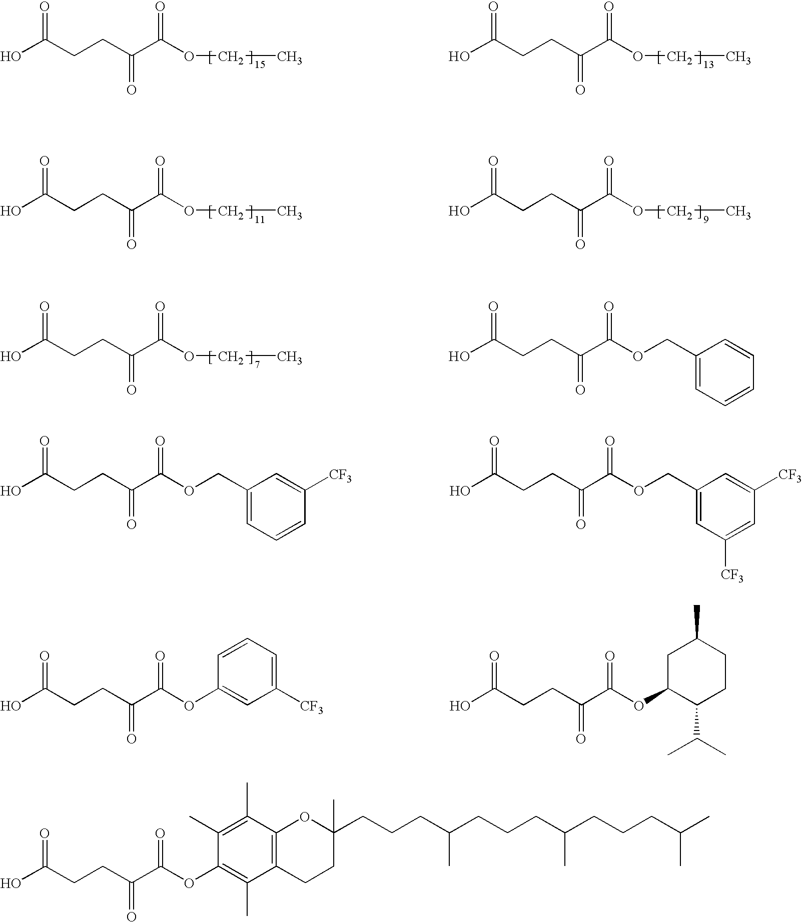

106. A compound according to claim 74, that:
activates HIFα hydroxylase; or
increases the level of α-ketoglutarate; or
activates HIFα hydroxylase; or
activates HIFα prolyl hydroxylase; or
increases the level of α-ketoglutarate.

107. A pharmaceutical composition comprising a compound according to claim 74, and a pharmaceutically acceptable carrier.

108. A method of activating PHD in a cell, in vitro or in vivo, comprising contacting the cell with an effective amount of a compound according to claim 74.

109. A method of inhibiting or preventing HIF stabilization in a cell, in vitro or in vivo, comprising contacting the cell with an effective amount of a compound according to claim 74.

110. A method of activating HIFα hydroxylase in a cell, in vitro or in vivo, comprising contacting the cell with an effective amount of a compound according to claim 74.

111. A method of (a) regulating (e.g., inhibiting) cell proliferation (e.g., proliferation of a cell), (b) inhibiting cell cycle progression, (c) promoting apoptosis, or (d) a combination of one or more these, in vitro or in vivo, comprising contacting cells (or the cell) with an effective amount of a compound according to claim 74.

112. A method of treatment of a condition, comprising administering to a patient in need of treatment a therapeutically effective amount of a compound according to claim 74, wherein the condition is selected from:
a condition that encounters hypoxic conditions as it proceeds;
a condition that is characterised by inappropriate, excessive, and/or undesirable angiogenesis;
a condition characterised by hypoxia-induced angiogenesis;
angiogenesis in which the activity of HIF-1α is upregulated due to hypoxia;
a condition selected from: cancer, psoriasis, atherosclerosis, menorrhagia, endometrosis, arthritis (both inflammatory and rheumatoid), macular degeneration, Paget's disease, retinopathy and its vascular complications (including proliferative and diabetic retinopathy), benign vascular proliferation, fibroses, obesity and inflammation;
a proliferative condition;
cancer;
solid tumour cancer;
cancer selected from: phaeochromocytoma, paraganglioma, leiomyoma, renal cell carcinoma, gastric carcinoma, and colorectal carcinoma;
cancer characterised by SDH dysfunction;
cancer that develops SDH down-regulation in a later stage of the disease;
gastric or colorectal cancer;
Dukes' stage C of colorectal cancer;
oral carcinoma tumours;
cancer in which the activity of HIF-1α is upregulated due to hypoxia; and
cancer in which the activity of one of the enzymes of the TCA cycle is down-regulated.

113. A method of treatment of a condition as defined in claim 112, comprising co-administering to a patient in need of treatment: (a) a therapeutically effective amount of a first agent that is a compound according to claim 74, and (b) a second agent.

114. A method according to claim 113, wherein the first and second agents are administered separately, sequentially, or simultaneously.

115. A method according to claim 113, wherein the second agent is a compound that is an enhancer of aminolaevulinic acid (ALA) synthase.

116. A method according to claim 113, wherein the second agent is selected from: barbiturates, anticonvulsants, non-narcotic analgetics, and non-steriodal anti-inflammatory compounds.

117. A method according to claim 113, wherein the second agent is selected from: Allyl isopropyl acetamide, Phenobarbital, Deferoxamine, Felbamate, Lamotrigine, Tiagabine, Cyclophosphamide, N-methylprotoporphyrin, Succinyl-acetone, Carbamazepine, Ethanol, Phenyloin, Azapropazone, Chloroquine, Paracetamol, Griseofulvin, Cadmium, Iron, Pyridoxine.

118. A method according to claim 113, wherein the second agent is selected from: Ethosuximide, Diazepam, Hydantoins, Methsuximide, Paramethadione, Phenobarbitone, Phensuximide, Phenyloin, Primidone, Succinimides, Bromides, Aspirin, Dihydroergotamine-Mesylate, Ergotamine Tartrate, Chloramphenicol, Dapsone, Erythromycin, Flucloxacillin, Pyrazinamide, Sulphonamides, Ampicillin, Vancomycin, Sulphonylureas Glipizidelnsulin, Alpha tocopheryl acetate, Ascorbic Acid, Folic Acid, Fructose, Glucose, Haem Arginate, Amidopyrine, Dichloralphenazone, Diclofenac Na, Dipyrone, Oxyphenbutazone, Propyphenazone, Aspitin, Codeine PO4, Dihydrocodeine, Canthaxanthin, β Carotene.

119. A method according to claim 112, further comprising the step of subjecting the patient to photodynamic therapy.

120. A method of treatment of a condition, comprising the steps of:
(i) simultaneous, separate, or sequential administration of: (a) a first agent, that is a compound according to claim 74; and (b) a photosensitizer; followed by
(ii) light irradiation;
wherein the condition is selected from:
a condition that encounters hypoxic conditions as it proceeds;
a condition that is characterised by inappropriate, excessive, and/or undesirable angiogenesis;
a condition characterised by hypoxia-induced angiogenesis;
angiogenesis in which the activity of HIF-1α is upregulated due to hypoxia;
a condition selected from: cancer, psoriasis, atherosclerosis, menorrhagia, endometrosis, arthritis (both inflammatory and rheumatoid), macular degeneration, Paget's disease, retinopathy and its vascular complications (including proliferative and diabetic retinopathy), benign vascular proliferation, fibroses, obesity and inflammation;
a proliferative condition;
cancer;
solid tumour cancer;
cancer selected from: phaeochromocytoma, paraganglioma, leiomyoma, renal cell carcinoma, gastric carcinoma, and colorectal carcinoma;
cancer characterised by SDH dysfunction;
cancer that develops SDH down-regulation in a later stage of the disease;
gastric or colorectal cancer;
Dukes' stage C of colorectal cancer;
oral carcinoma tumours;
cancer in which the activity of HIF-1α is upregulated due to hypoxia; and
cancer in which the activity of one of the enzymes of the TCA cycle is down-regulated.

121. A kit comprising: (a) a compound according to claim 74; and (b) instructions for use.
